(19) United States
(12) Patent Application Publication
(10) Pub. No.: US 2013/0065811 A1
(43) Pub. Date: Mar. 14, 2013

(54) STABLE WATER-SOLUBLE UNIT DOSE ARTICLE

(71) Applicants: Susana FERNANDEZ-PRIETO, Castellon (ES); Isabelle GUIMET, Brussels (BE); Regine LABEQUE, Heembeek (BE); Walter August Maria BROECKX, Berlare (BE); John SMETS, Lubbeek (BE)

(72) Inventors: Susana FERNANDEZ-PRIETO, Castellon (ES); Isabelle GUIMET, Brussels (BE); Regine LABEQUE, Heembeek (BE); Walter August Maria BROECKX, Berlare (BE); John SMETS, Lubbeek (BE)

(21) Appl. No.: 13/602,584

(22) Filed: Sep. 4, 2012

(30) Foreign Application Priority Data
Sep. 13, 2011 (EP) 11 181 102.2

(51) Int. Cl. C11D 17/00; A61K 8/00

(52) U.S. Cl. 510/375; 510/393; 510/406; 264/4

(57) ABSTRACT

The need for a means to formulate fluid compositions for use in water-soluble unit dose articles having higher levels of water is met by incorporating a di-amido gellant into the fluid composition. The di-amido gellant also simplifies the addition of ingredients to water-soluble unit dose articles, such as microcapsules, that are typically added as aqueous suspensions or slurries.

### FIELD OF THE INVENTION

The present invention relates water-soluble unit dose articles that are stable even when they comprise high water levels.

### BACKGROUND OF THE INVENTION

Today's consumers desire easy to use, convenient, products for a variety of applications, including treating fabrics and hard surfaces. A suitable means of delivering such treatments is by encompassing a fluid composition, which delivers the treatment benefit, in a water-soluble film, to form a water-soluble unit dose article. However, in order to prevent the fluid composition from “sweating” through the water-soluble film, or opening the seals of the unit dose article, or even dissolving the water soluble film material, the water level in the composition has to be strictly limited.

Thus, when formulating a fluid composition to be encapsulated in a water soluble film, anhydrous, or low water premixes of ingredients must be used. This adds both cost and complexity to the making operation. In addition, there are many ingredients that are challenging to supply as anhydrous or low water premixes. For instance, microcapsules are typically formed via emulsion polymerisation, and hence are incorporated as aqueous suspensions comprising excess water. Such ingredients are therefore either added in very limited amounts, or are omitted.

Therefore, a need remains for a means to formulate fluid compositions for use in water-soluble unit dose articles having higher levels of water.

### SUMMARY OF THE INVENTION

According to the present invention, there is provided a unit dose article comprising a water soluble film encapsulating a fluid composition, wherein the fluid composition comprises: a di-amido gellant; and from 11 wt % to 70 wt % by weight of water. The present invention also provides for a process of making a unit dose article comprising the steps of: providing a di-amido gellant premix comprising a di-amido gellant and a solvent; combining the di-amido gellant premix with a fluid feed, wherein the fluid feed comprises from 10% to 70% by weight water, to form a fluid composition; and encapsulating the fluid composition in a water soluble film.

### DETAILED DESCRIPTION OF THE INVENTION

The unit dose article of the present invention comprises a water-soluble film which fully encloses a fluid composition in at least one compartment. Suitable fluid compositions include, but are not limited to, consumer products such as: products for treating fabrics, hard surfaces and any other surfaces in the area of fabric and home care, including: dishwashing, laundry cleaning, laundry and rinse additives, and hard surface cleaning including floor and toilet bowl cleaners. A particularly preferred embodiment of the invention is a “fluid laundry treatment composition”. As used herein, “fluid laundry treatment composition” refers to any laundry treatment composition comprising a fluid capable of wetting and treating fabric e.g., cleaning clothing in a domestic washing machine.

The fluid composition can include solids or gases in suitably subdivided form, but the fluid composition excludes forms which are non-fluid overall, such as tablets or granules. The fluid compositions preferably have densities in the range from of 0.9 to 1.3 grams per cubic centimeter, more preferably from 1.00 to 1.1 grams per cubic centimeter, excluding any solid additives, but including any bubbles, if present.

All percentages, ratios and proportions used herein are by weight percent of the fluid composition, unless otherwise specified. All average values are calculated “by weight” of the composition or components thereof, unless otherwise expressly indicated.

### The Unit Dose Article

The unit dose article can be of any form, shape and material which is suitable for holding the fluid composition, i.e. without allowing the release of the fluid composition, and any additional component, from the unit dose article prior to contact of the unit dose article with water. The exact execution will depend, for example, on the type and amount of the compositions in the unit dose article, the number of compartments in the unit dose article, and on the characteristics required from the unit dose article to hold, protect and deliver or release the compositions or components.

The unit dose article comprises a water-soluble film which fully encloses the fluid composition in at least one compartment. The unit dose article may optionally comprise additional compartments; said additional compartments may comprise an additional composition. Said additional composition may be fluid, solid, and mixtures thereof. Alternatively, any additional solid component may be suspended in a fluid-filled compartment. A multi-compartment unit dose form may be desirable for such reasons as: separating chemically incompatible ingredients; or where it is desirable for a portion of the ingredients to be released into the wash earlier or later.

Water-soluble film: The water-soluble film typically has a solubility of at least 50%, preferably at least 75%, more preferably at least 95%. The method for determining water-solubility of the film is given in the Test Methods. The water-soluble film typically has a dissolution time of less than 100 seconds, preferably less than 85 seconds, more preferably less than 75 seconds, most preferably less than 60 seconds. The method for determining the dissolution time of the film is given in the Test Methods.

Preferred films are polymeric materials, preferably polymers which are formed into a film or sheet. The film can be obtained by casting, blow-moulding, extrusion or blow extrusion of the polymer material, as known in the art. Preferably, the water-soluble film comprises: polymers, copolymers or derivatives thereof, including polyvinyl alcohols (PVA), polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthan gum and carragum, and mixtures thereof. More preferably, the water-soluble film comprises: polyacrylates and water-soluble acrylate copolymers, methylcellulose, carboxymethylcellulose, dextrin, ethylcellulose, hydroxyethyl cellulose, hydroxypropyl methylcellulose, maltodextrin, polymethacrylates, and mixtures thereof. Most preferably, the water-soluble film comprises: polyvinyl alcohols, polyvinyl alcohol copolymers, hydroxypropyl methyl cellulose (HPMC), and mixtures thereof. Preferably, the level of polymer or copolymer in the film is at least 60% by weight. The polymer or copolymer preferably has a weight average molecular weight of from 1000 to 1,000,000, more preferably from 10,000 to 300,000, even more preferably form 15,000 to 200,000, and most preferably from 20,000 to 150,000 g/mol.

Copolymers and mixtures of polymers can also be used. This may in particular be beneficial to control the mechanical and/or dissolution properties of the compartments or unit dose article, depending on the application thereof and the required needs. For example, it may be preferred that a mixture of polymers is present in the film, whereby one polymer material has a higher water-solubility than another polymer material, and/or one polymer material has a higher mechanical strength than another polymer material. Using copolymers and mixtures of polymers can have other benefits, including improved long-term resiliency of the water-soluble or dispersible film to the fluid composition ingredients. For instance, U.S. Pat. No. 6,787,512 discloses polyvinyl alcohol copolymer films comprising a hydrolyzed copolymer of vinyl acetate and a second sulfonic acid monomer, for improved resiliency against detergent ingredients. An example of such a film is sold by Monosol of Merrillville, Ind., US, under the brand name: M8900. It may be preferred that a mixture of polymers is used, having different weight average molecular weights, for example a mixture of polyvinyl alcohol or a copolymer thereof, of a weight average molecular weight of from 10,000 to 40,000 g/mol, and of another polyvinyl alcohol or copolymer, with a weight average molecular weight of from 100,000 to 300,000 g/mol. U.S. 2011/0189413 discloses example of blend of polyvinyl alcohol with different molecular weight. An example of such a film is sold by MonoSol under the brand name M8779.

Also useful are polymer blend compositions, for example comprising hydrolytically degradable and water-soluble polymer blends such as polylactide and polyvinyl alcohol, achieved by the mixing of polylactide and polyvinyl alcohol, typically comprising 1 to 35% by weight of the film of polylactide, and from 65% to 99% by weight of polyvinyl alcohol. The polymer present in the film may be from 60% to 98% hydrolysed, more preferably from 80% to 90%, to improve the dissolution/dispersion of the film material.

The water-soluble film herein may comprise additive ingredients other than the polymer or copolymer material. For example, it may be beneficial to add: plasticisers such as glycerol, ethylene glycol, diethyleneglycol, propylene glycol, sorbitol and mixtures thereof; additional water; and/or disintegrating aids.

Other suitable examples of commercially available water-soluble films include polyvinyl alcohol and partially hydrolysed polyvinyl acetate, alginates, cellulose ethers such as carboxymethylcellulose and methylcellulose, polyethylene oxide, polyacrylates and combinations of these. Most preferred are films with similar properties to the polyvinyl alcohol comprising film known under the trade reference M8630, sold by Monosol of Merrillville, Ind., US.

### Fluid Compositions:

As used herein, “fluid composition” refers to fluid compositions comprising from 11% to 70%, preferably from 13% to 50%, more preferably 15% to 35%, even more preferably 17% to 30%, most preferably from 20% to 25% by weight of water.

The fluid composition of the present invention may also comprise from 2% to 40%, more preferably from 5% to 25% by weight of a non-aqueous solvent. Preferably, the non-aqueous solvent is fluid at ambient temperature and pressure (i.e. 21° C. and 1 atmosphere). Preferred non-aqueous solvents are organic solvents which contain no amino functional groups. Preferred non-aqueous solvents are selected from the group consisting of: monohydric alcohols, dihydric alcohols, polyhydric alcohols, glycerol, glycols including polyalkylene glycols such as polyethylene glycol, and mixtures thereof. More preferred non-aqueous solvents include monohydric alcohols, dihydric alcohols, polyhydric alcohols, glycerol, and mixtures thereof. Highly preferred are mixtures of solvents, especially mixtures of two or more of the following: lower aliphatic alcohols, diols, and glycerols. Preferred lower aliphatic alcohols are ethanol, propanol, butanol, isopropanol, and mixtures thereof. Preferred diols are 1,2-propanediol or 1,3-propanediol, and mixtures thereof. Also preferred are propanediol and mixtures thereof with diethylene glycol, where the mixture contains no methanol or ethanol. Thus embodiments of fluid compositions of the present invention may include embodiments in which propanediols are used but methanol and ethanol are not used. Non-aqueous solvents may be present when preparing a premix, or in the final fluid composition.

### Di-amido-gellants:

The fluid composition comprises a di-amido gellant, preferably at a level of from 0.01 wt % to 10 wt %, preferably from 0.05 wt % to 5 wt %, more preferably from 0.075 wt % to 2 wt %, most preferably from 0.1 wt % to 0.5 wt % of the di-amido gellant.

Di-amido gellants comprise at least two nitrogen atoms, wherein at least two of said nitrogen atoms form amido functional groups. The di-amido gellant preferably has the following formula:

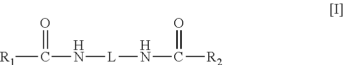

wherein: R₁ and R₂ are aminofunctional end-groups which may be the same or different and L is a linking moeity of molecular weight from 14 to 500 g/mol. An aminofunctional end-group is one that comprises a nitrogen atom. The linking moiety, L, can be any suitable group that connects the amido functional groups together. By suitably selecting the linking moiety, L, the separation of the amido functional groups can be adjusted.

Preferably, the di-amido gellant has a molecular weight from 150 to 1500 g/mol, more preferably from 300 g/mol to 900 g/mol, most preferably from 400 g/mol to 700 g/mol.

In a preferred embodiment: R₁ is R₃ or

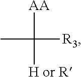

### and R₂ is R₄ or

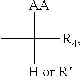

wherein AA is selected from the group consisting of:

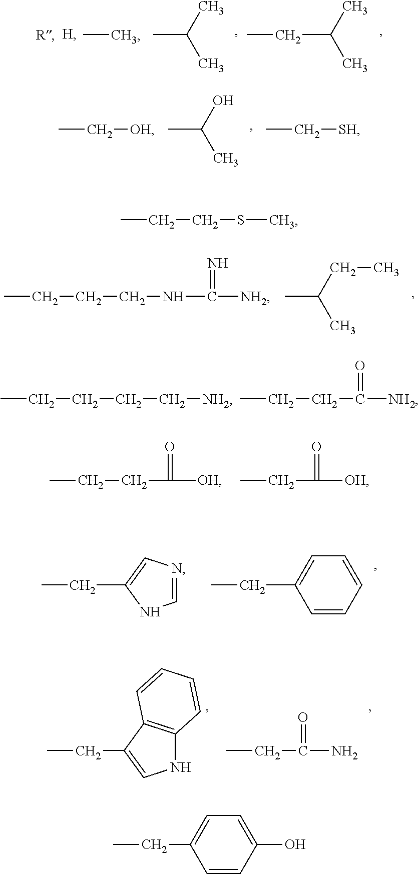

and R₃ and R₄ independently have the formula:

(L′)ₘ-(L″)_{q}-R [II],

where (m+q) is from 1 to 10.

However, for R₁, the combination of AA, R′, and R₃ must be selected such that R₁ is an aminofunctional end-group. Similarly, for R₂, the combination of AA, R′, and R₄ must be selected such that R₂ is an aminofunctional end-group.

Preferably, L has the formula:

Aₐ-B_{b}-C_{c}-D_{d} [III],

where (a+b+c+d) is from 1 to 20,
wherein L′, L″ from formula [II] and A, B, C, D from formula [III] are independently selected from the group consisting of:

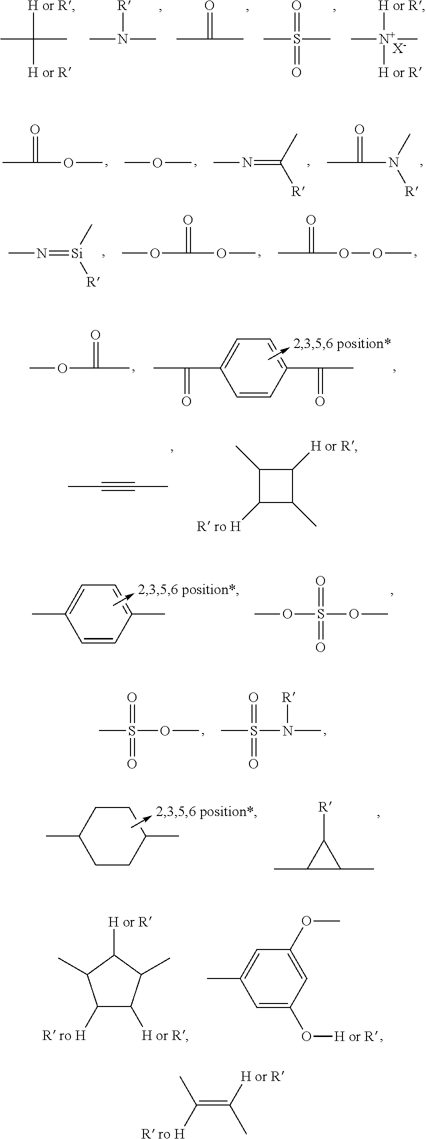

Preferably, L′, L″ from formula [II] and A, B, C, D from formula [III] are independently selected from the group consisting of:

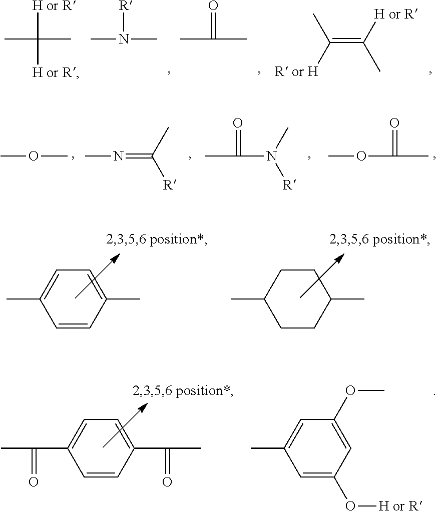

*the arrow indicates up to 4 substitutions in the positions indicated, and X⁻ an anion and R, R′ and R″ are independently selected from AA and the group consisting of:

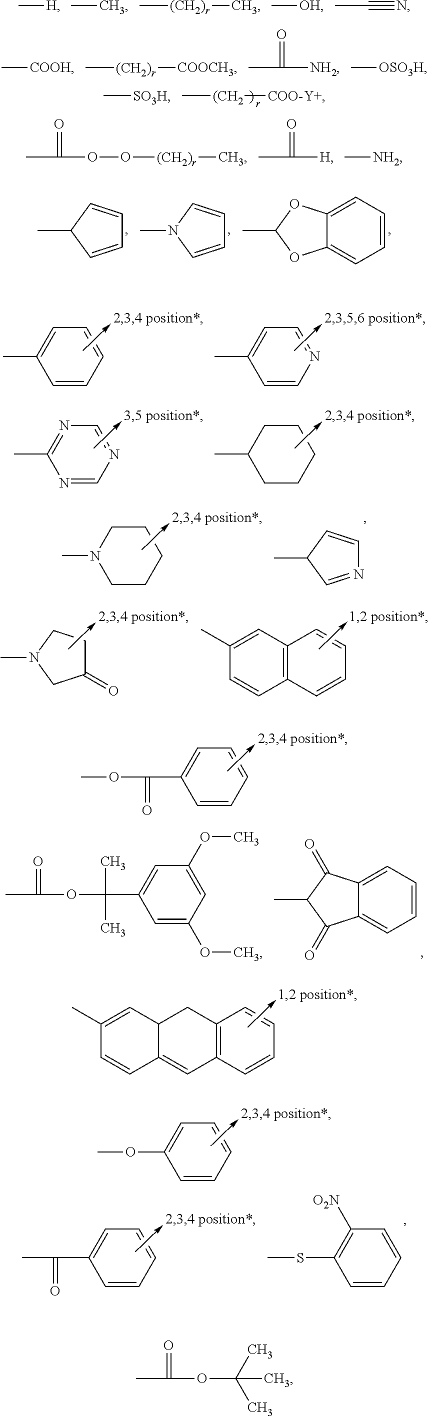

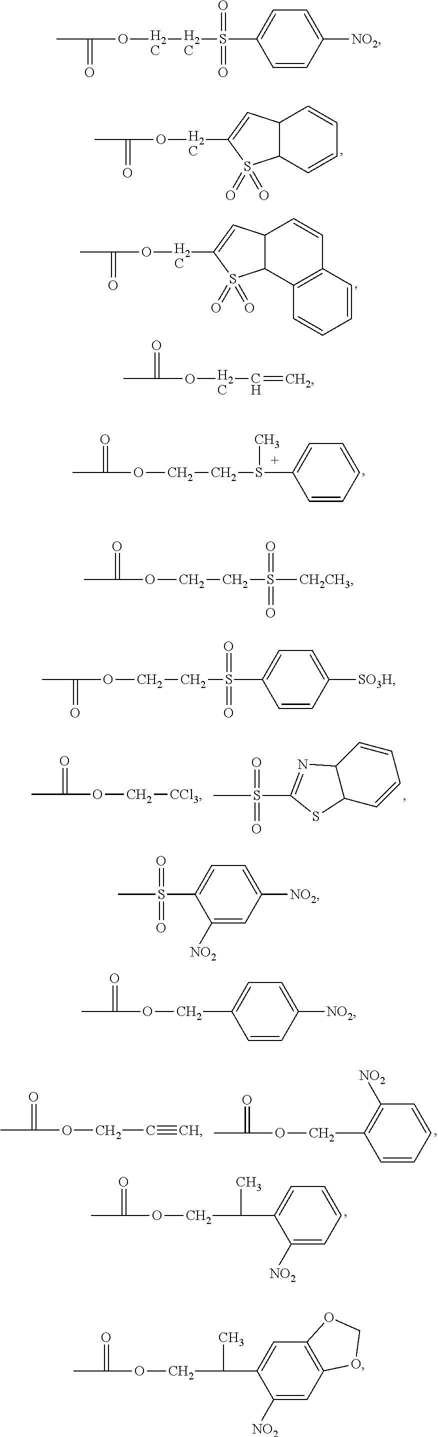

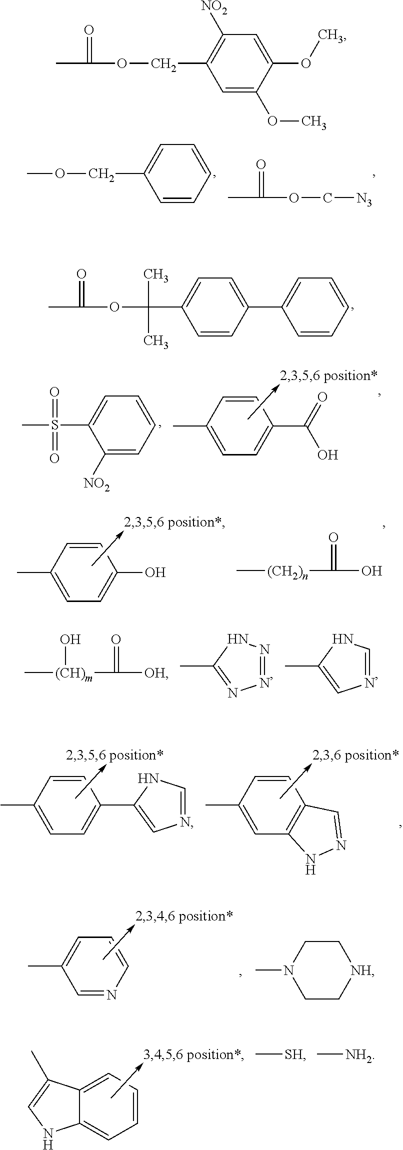

*the arrow indicates up to 4 substitutions in the positions indicated, r, m and n are integers from 1 to 20 and Y+ is a cation

Preferably, R, R′ and R″ are independently selected from the group consisting of:

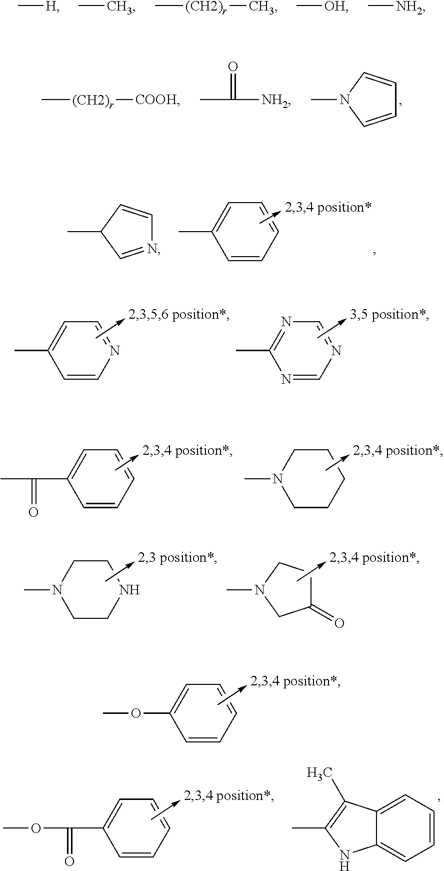

In a more preferred embodiment, the di-amido gellant is characterized in that:

L is an aliphatic linking group with a backbone chain of from 2 to 20 carbon atoms, preferably —(CH₂)ₙ— wherein n is selected from 2 to 20. Preferably, R₁ and R₂ both have the structure:

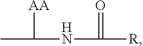

wherein: AA is selected from the group consisting of:

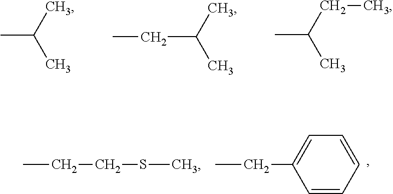

and R is selected from the group:

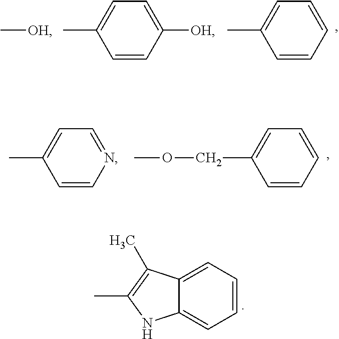

In another embodiment R, R′ and R″ can independently be selected from the group consisting of: an ethoxy group, an epoxy group with 1 to 15 ethoxy or epoxy units. In another embodiment, the R, R′ and R″ may comprise a functional end group selected from the group consisting of: an aromatic, alicyclic, heteroaromatic, heterocyclic group including mono-, di-, and oligo-polysaccharides.

In another embodiment, two or more of L, L′ and L″ are the same group. The di-amido gellant molecule can be symmetric with respect to the L entity or can be asymmetric. Without intending to be bound by theory, it is believed that symmetric di-amido gellant molecules allow for more orderly structured networks to form, and are hence more efficient at sequestering water and providing structuring. In contrast, compositions comprising one or more asymmetric di-amido gellant molecules can create less ordered networks.

In one embodiment, the AA comprises at least one of: Alanine, β-Alanine and substituted Alanines; Linear Amino-Alkyl Carboxylic Acid; Cyclic Amino-Alkyl Carboxylic Acid; Aminobenzoic Acid Derivatives; Aminobutyric Acid Derivatives; Arginine and Homologues; Asparagine; Aspartic Acid; p-Benzoyl-Phenylalanine; Biphenylalanine; Citrulline; Cyclopropylalanine; Cyclopentylalanine; Cyclohexylalanine; Cysteine, Cystine and Derivatives; Diaminobutyric Acid Derivatives; Diaminopropionic Acid; Glutamic Acid Derivatives; Glutamine; Glycine; Substituted Glycines; Histidine; Homoserine; Indole Derivatives; Isoleucine; Leucine and Derivatives; Lysine; Methionine; Naphthylalanine; Norleucine; Norvaline; Ornithine; Phenylalanine; Ring-Substituted Phenylalanines; Phenylglycine; Pipecolic Acid, Nipecotic Acid and Isonipecotic Acid; Proline; Hydroxyproline; Thiazolidine; Pyridylalanine; Serine; Statine and Analogues; Threonine; Tetrahydronorharman-3-carboxylic Acid; 1,2,3,4-Tetrahydroisoquinoline; Tryptophane; Tyrosine; Valine; and combinations thereof.

In one embodiment, the di-amido gellant comprises a pH tuneable group, to result in a pH-tuneable di-amido gellant. A pH tuneable di-amido gellant can provide the fluid composition with a viscosity profile that changes with the pH of the composition. Hence, a pH tuneable di-amido gellant can be added to a fluid composition at a pH at which the viscosity is sufficiently low to allow easy mixing, before changing the pH such that the pH tuneable di-amido gellant provides structuring.

The pH tuneable di-amido gellants comprise at least one pH sensitive group, that is either protonated or deprotonated by a change in composition pH. When a pH tuneable di-amido gellant is added to a fluid composition comprising water, it is believed that the uncharged form of the di-amido gellant builds viscosity while the charged form is more soluble and less efficient at forming a viscosity building network. By increasing or decreasing the pH (depending on the selection of the pH-sensitive groups) the di-amido gellant is either protonated or deprotonated. Thus, by changing the pH of the solution, the solubility, and hence the viscosity building behaviour, of the di-amido gellant can be controlled. By careful selection of the pH-sensitive groups, the pKa of the di-amido gellant can be tailored. Hence, the choice of the pH-sensitive groups can be used to select the pH at which the di-amido gellant builds viscosity.

In one embodiment, L, R₁, R₂, and mixtures thereof, may comprise the pH tuneable group. In a preferred embodiment, R₁ and R₂ comprise the pH-tuneable group. In another embodiment R, R′ and R″ are amino functional end-groups, preferably amido functional end-group, more preferably pH-tuneable amido functional groups. In a preferred embodiment, the pH tuneable group comprises at least one pyridine group. Preferably, di-amido gellant comprises a pH tuneable group, such that the di-amido gellant has a pKa of from 0 to 30, more preferably from 1.5 to 14, even more preferably from 3 to 9, even more preferably from 4 to 8.

It is believed that di-amido gellants are able to sequester water and hence prevent the water from interacting with other ingredients, such as the water soluble film. Thus, the di-amido gellants enable higher water containing fluid compositions to be enclosed in a water-soluble film, without causing film dissolution, or film sweating.

The di-amido-gellants can also be used for improving the structuring of the fluid composition and for suspending ingredients such as particulates in the fluid composition. Preferably, the fluid composition comprising the di-amido-gellant has a resting viscosity (see Test Methods) of at least 1,000 cps, more preferably at least 10,000 cps, most preferably at least 50,000 cps. This resting (low stress) viscosity represents the viscosity of the fluid composition under gentle shaking in the unit-dose article, such as during transportation.

To provide more robust structuring, the fluid detergent may comprise a mixture of two or more di-amido gellants. Such a mixture may include a di-amido gellant which has a higher solubility in water, with a di-amido gellant with higher solubility in non-aminofunctional solvents. Without intending to be bound by theory, it is believed that combining a di-amido gellant that is more soluble in water with a di-amido gellant that is more soluble in non-aminofunctional solvents provides improved structuring and stability to the formula. A preferred combination is: N,N′-(2S,2′S)-1,1′-(dodecane-1,12-diylbis(azanediyl))bis(3-methyl-1-oxobutane-2,1-diyl)diisonicotinamide with the more water-soluble N,N′-(2S,2′S)-1,1′-(propane-1,3-diylbis(azanediyl))bis(3-methyl-1-oxobutane-2,1-diyl)diisonicotinamide.

The di-amido gellant molecules may also comprise protective groups, preferably from 1 to 2 protective groups, most preferably two protective groups. Examples of suitable protective groups are provided in “Protecting Groups”, P. J. Kocienski, ISBN 313 135601 4, Georg Thieme Verlag, Stutgart; and “Protective Groups in Organic Chemistry”, T. W. Greene, P. G. M. Wuts, ISBN 0-471-62301-6, John Wiley & Sons, Inc, New York.

The di-amido gellant preferably has a minimum gelling concentration (MGC) of from 0.1 to 100 mg/mL in the fluid composition, preferably from 0.1 to 25 mg/mL, more preferred from 0.5 to 10 mg/mL in accordance with the MGC Test Method. The MGC as used herein can be represented as mg/ml or as a wt %, where wt % is calculated as the MGC in mg/ml divided by 10. In one embodiment, when measured in the fluid composition, the MGC is from 0.1 to 100 mg/mL, preferably from 0.1 to 25 mg/mL of said di-amido gellant, more preferably from 0.5 to 10 mg/mL, or at least 0.1 mg/mL, at least 0.3 mg/mL, at least 0.5 mg/mL, at least 1.0 mg/mL, at least 2.0 mg/mL, at least 5.0 mg/mL of di-amido gellant. While the invention includes fluid compositions having a di-amido gellant concentration either above or below the MGC, the di-amido gellants of the invention result in particularly useful rheologies below the MGC.

Suitable di-amido gellants, and mixtures thereof, may be selected from table 1:

TABLE 1
Non-limiting examples of di-amido gellants of use in unit dose articles of the present invention:

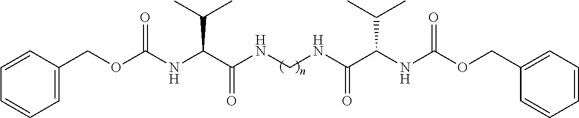

| | |
|---|---|
| dibenzyl (2S,2′S)-1,1′-(ethane-1,2- | dibenzyl (2S,2′S)-1,1′-(propane-1,3- |
| diylbis(azanediyl))bis(3-methyl-1-oxobutane- | diylbis(azanediyl))bis(3-methyl-1-oxobutane- |
| 2,1-diyl)dicarbamate | 2,1-diyl)dicarbamate |
| dibenzyl (2S,2′S)-1,1′-(butane-1,4- | dibenzyl (2S,2′S)-1,1′-(pentane-1,5- |
| diylbis(azanediyl))bis(3-methyl-1-oxobutane- | diylbis(azanediyl))bis(3-methyl-1-oxobutane- |
| 2,1-diyl)dicarbamate | 2,1-diyl)dicarbamate |
| dibenzyl (2S,2′S)-1,1′-(hexane-1,6- | dibenzyl (2S,2′S)-1,1′-(heptane-1,7- |
| diylbis(azanediyl))bis(3-methyl-1-oxobutane- | diylbis(azanediyl))bis(3-methyl-1-oxobutane- |
| 2,1-diyl)dicarbamate | 2,1-diyl)dicarbamate |
| dibenzyl (2S,2′S)-1,1′-(octane-1,8- | dibenzyl (2S,2′S)-1,1′-(nonane-1,9- |
| diylbis(azanediyl))bis(3-methyl-1-oxobutane- | diylbis(azanediyl))bis(3-methyl-1-oxobutane- |
| 2,1-diyl)dicarbamate | 2,1-diyl)dicarbamate |
| dibenzyl (2S,2′S)-1,1′-(decane-1,10- | dibenzyl (2S,2′S)-1,1′-(undecane-1,11- |
| diylbis(azanediyl))bis(3-methyl-1-oxobutane- | diylbis(azanediyl))bis(3-methyl-1-oxobutane- |
| 2,1-diyl)dicarbamate | 2,1-diyl)dicarbamate |
| dibenzyl (2S,2′S)-1,1′-(dodecane-1,12- | dibenzyl (2S,2′S)-1,1′-(tridecane-1,13- |
| diylbis(azanediyl))bis(3-methyl-1-oxobutane- | diylbis(azanediyl))bis(3-methyl-1-oxobutane- |
| 2,1-diyl)dicarbamate | 2,1-diyl)dicarbamate |
| dibenzyl (2S,2′S)-1,1′-(hexadecane-1,16- | dibenzyl (2S,2′S)-1,1′-(octodecane-1,18- |
| diylbis(azanediyl))bis(3-methyl-1-oxobutane- | diylbis(azanediyl))bis(3-methyl-1-oxobutane- |
| 2,1-diyl)dicarbamate | 2,1-diyl)dicarbamate |
| dibenzyl (2S,2′S)-1,1′-(tetradecane-1,14- | |
| diylbis(azanediyl))bis(3-methyl-1-oxobutane- | |
| 2,1-diyl)dicarbamate | |

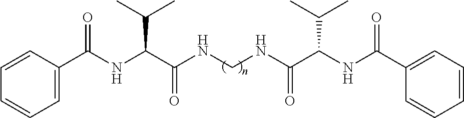

| | |
|---|---|
| N,N′-(2S,2′S)-1,1′-(ethane-1,2- | N,N′-(2S,2′S)-1,1′-(butane-1,4- |
| diylbis(azanediyl))bis(3-methyl-1-oxobutane- | diylbis(azanediyl))bis(3-methyl-1-oxobutane- |
| 2,1-diyl)dibenzamide | 2,1-diyl)dibenzamide |
| N,N′-(2S,2′S)-1,1′-(propane-1,3- | N,N′-(2S,2′S)-1,1′-(hexane-1,6- |
| diylbis(azanediyl))bis(3-methyl-1-oxobutane- | diylbis(azanediyl))bis(3-methyl-1-oxobutane- |
| 2,1-diyl)dibenzamide | 2,1-diyl)dibenzamide |
| N,N′-(2S,2′S)-1,1′-(pentane-1,5- | N,N′-(2S,2′S)-1,1′-(octane-1,8- |
| diylbis(azanediyl))bis(3-methyl-1-oxobutane- | diylbis(azanediyl))bis(3-methyl-1-oxobutane- |
| 2,1-diyl)dibenzamide | 2,1-diyl)dibenzamide |
| N,N′-(2S,2′S)-1,1′-(heptane-1,7- | -(2S,2′S)-1,1′-(decane-1,10- |
| diylbis(azanediyl))bis(3-methyl-1-oxobutane- | diylbis(azanediyl))bis(3-methyl-1-oxobutane- |
| 2,1-diyl)dibenzamide | 2,1-diyl)dibenzamide |
| N,N′-(2S,2′S)-1,1′-(nonane-1,9- | N,N′-(2S,2′S)-1,1′-(dodecane-1,12- |
| diylbis(azanediyl))bis(3-methyl-1-oxobutane- | diylbis(azanediyl))bis(3-methyl-1-oxobutane- |
| 2,1-diyl)dibenzamide | 2,1-diyl)dibenzamide |
| N,N′-(2S,2′S)-1,1′-(undecane-1,11- | N,N′-(2S,2′S)-1,1′-(hexadecane-1,16- |
| diylbis(azanediyl))bis(3-methyl-1-oxobutane- | diylbis(azanediyl))bis(3-methyl-1-oxobutane- |
| 2,1-diyl)dibenzamide | 2,1-diyl)dibenzamide |
| N,N′-(2S,2′S)-1,1′-(tridecane-1,13- | N,N′-(2S,2′S)-1,1′-(tetradecane-1,14- |
| diylbis(azanediyl))bis(3-methyl-1-oxobutane- | diylbis(azanediyl))bis(3-methyl-1-oxobutane- |
| 2,1-diyl)dibenzamide | 2,1-diyl)dibenzamide |
| N,N′-(2S,2′S)-1,1′-(octadecane-1,18- | |
| diylbis(azanediyl))bis(3-methyl-1-oxobutane- | |
| 2,1-diyl)dibenzamide | |

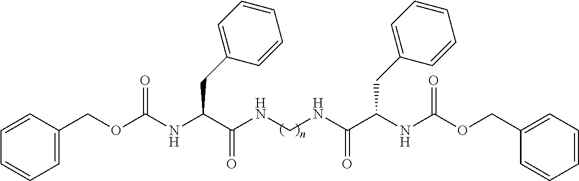

| | |
|---|---|
| dibenzyl (2S,2′S)-1,1′-(propane-1,3- | dibenzyl (2S,2′S)-1,1′-(ethane-1,2- |
| diylbis(azanediyl))bis(1-oxo-3- | diylbis(azanediyl))bis(1-oxo-3-phenylpropane- |
| phenylpropane-2,1-diyl)dicarbamate | 2,1-diyl)dicarbamate |
| dibenzyl (2S,2′S)-1,1′-(pentane-1,5- | dibenzyl (2S,2′S)-1,1′-(butane-1,4- |
| diylbis(azanediyl))bis(1-oxo-3- | diylbis(azanediyl))bis(1-oxo-3-phenylpropane- |
| phenylpropane-2,1-diyl)dicarbamate | 2,1-diyl)dicarbamate |
| dibenzyl (2S,2′S)-1,1′-(heptane-1,7- | dibenzyl (2S,2′S)-1,1′-(hexane-1,6- |
| diylbis(azanediyl))bis(1-oxo-3- | diylbis(azanediyl))bis(1-oxo-3-phenylpropane- |
| phenylpropane-2,1-diyl)dicarbamate | 2,1-diyl)dicarbamate |
| dibenzyl (2S,2′S)-1,1′-(decane-1,10- | dibenzyl (2S,2′S)-1,1′-(nonane-1,9- |
| diylbis(azanediyl))bis(1-oxo-3- | diylbis(azanediyl))bis(1-oxo-3-phenylpropane- |
| phenylpropane-2,1-diyl)dicarbamate | 2,1-diyl)dicarbamate |
| dibenzyl (2S,2′S)-1,1′-(dodecane-1,12- | dibenzyl (2S,2′S)-1,1′-(undecane-1,11- |
| diylbis(azanediyl))bis(1-oxo-3- | diylbis(azanediyl))bis(1-oxo-3-phenylpropane- |
| phenylpropane-2,1-diyl)dicarbamate | 2,1-diyl)dicarbamate |
| dibenzyl (2S,2′S)-1,1′-(hexadecane-1,16- | dibenzyl (2S,2′S)-1,1′-(tridecane-1,13- |
| diylbis(azanediyl))bis(1-oxo-3- | diylbis(azanediyl))bis(1-oxo-3-phenylpropane- |
| phenylpropane-2,1-diyl)dicarbamate | 2,1-diyl)dicarbamate |
| dibenzyl (2S,2′S)-1,1′-(tetradecane-1,14- | dibenzyl (2S,2′S)-1,1′-(octadecane-1,18- |
| diylbis(azanediyl))bis(1-oxo-3- | diylbis(azanediyl))bis(1-oxo-3-phenylpropane- |
| phenylpropane-2,1-diyl)dicarbamate | 2,1-diyl)dicarbamate |

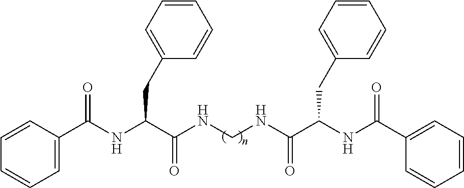

| | |
|---|---|
| N,N′-(2S,2′S)-1,1′-(ethane-1,2- | N,N′-(2S,2′S)-1,1′-(propane-1,3- |
| diylbis(azanediyl))bis(1-oxo-3- | diylbis(azanediyl))bis(1-oxo-3-phenylpropane- |
| phenylpropane-2,1-diyl)dibenzamide | 2,1-diyl)dibenzamide |
| N,N′-(2S,2′S)-1,1′-(butane-1,4- | N,N′-(2S,2′S)-1,1′-(pentane-1,5- |
| diylbis(azanediyl))bis(1-oxo-3- | diylbis(azanediyl))bis(1-oxo-3-phenylpropane- |
| phenylpropane-2,1-diyl)dibenzamide | 2,1-diyl)dibenzamide |
| N,N′-(2S,2′S)-1,1′-(hexane-1,6- | N,N′-(2S,2′S)-1,1′-(heptane-1,7- |
| diylbis(azanediyl))bis(1-oxo-3- | diylbis(azanediyl))bis(1-oxo-3-phenylpropane- |
| phenylpropane-2,1-diyl)dibenzamide | 2,1-diyl)dibenzamide |
| N,N′-(2S,2′S)-1,1′-(octane-1,8- | N,N′-(2S,2′S)-1,1′-(nonane-1,9- |
| diylbis(azanediyl))bis(1-oxo-3- | diylbis(azanediyl))bis(1-oxo-3-phenylpropane- |
| phenylpropane-2,1-diyl)dibenzamide | 2,1-diyl)dibenzamide |
| N,N′-(2S,2′S)-1,1′-(decane-1,10- | N,N′-(2S,2′S)-1,1′-(undecane-1,11- |
| diylbis(azanediyl))bis(1-oxo-3- | diylbis(azanediyl))bis(1-oxo-3-phenylpropane- |
| phenylpropane-2,1-diyl)dibenzamide | 2,1-diyl)dibenzamide |
| N,N′-(2S,2′S)-1,1′-(dodecane-1,12- | N,N′-(2S,2′S)-1,1′-(tridecane-1,13- |
| diylbis(azanediyl))bis(1-oxo-3- | diylbis(azanediyl))bis(1-oxo-3-phenylpropane- |
| phenylpropane-2,1-diyl)dibenzamide | 2,1-diyl)dibenzamide |
| N,N′-(2S,2′S)-1,1′-(tetradecane-1,14- | N,N′-(2S,2′S)-1,1′-(hexadecane-1,16- |
| diylbis(azanediyl))bis(1-oxo-3- | diylbis(azanediyl))bis(1-oxo-3-phenylpropane- |
| phenylpropane-2,1-diyl)dibenzamide | 2,1-diyl)dibenzamide |
| N,N′-(2S,2′S)-1,1′-(octadecane-1,18- | |
| diylbis(azanediyl))bis(1-oxo-3- | |
| phenylpropane-2,1-diyl)dibenzamide | |

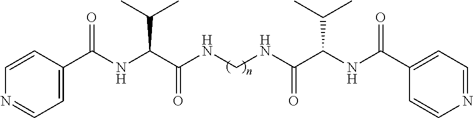

| | |
|---|---|
| N,N′-(2S,2′S)-1,1′-(ethane-1,2- | N,N′-(2S,2′S)-1,1′-(propane-1,3- |
| diylbis(azanediyl))bis(3-methyl-1-oxobutane- | diylbis(azanediyl))bis(3-methyl-1-oxobutane- |
| 2,1-diyl)diisonicotinamide | 2,1-diyl)diisonicotinamide |
| N,N′-(2S,2′S)-1,1′-(butane-1,4- | N,N′-(2S,2′S)-1,1′-(pentane-1,5- |
| diylbis(azanediyl))bis(3-methyl-1-oxobutane- | diylbis(azanediyl))bis(3-methyl-1-oxobutane- |
| 2,1-diyl)diisonicotinamide | 2,1-diyl)diisonicotinamide |
| N,N′-(2S,2′S)-1,1′-(hexane-1,6- | N,N′-(2S,2′S)-1,1′-(heptane-1,7- |
| diylbis(azanediyl))bis(3-methyl-1-oxobutane- | diylbis(azanediyl))bis(3-methyl-1-oxobutane- |
| 2,1-diyl)diisonicotinamide | 2,1-diyl)diisonicotinamide |
| N,N′-(2S,2′S)-1,1′-(octane-1,8- | N,N′-(2S,2′S)-1,1′-(nonane-1,9- |
| diylbis(azanediyl))bis(3-methyl-1-oxobutane- | diylbis(azanediyl))bis(3-methyl-1-oxobutane- |
| 2,1-diyl)diisonicotinamide | 2,1-diyl)diisonicotinamide |
| N,N′-(2S,2′S)-1,1′-(decane-1,10- | N,N′-(2S,2′S)-1,1′-(undecane-1,11- |
| diylbis(azanediyl))bis(3-methyl-1-oxobutane- | diylbis(azanediyl))bis(3-methyl-1-oxobutane- |
| 2,1-diyl)diisonicotinamide | 2,1-diyl)diisonicotinamide |
| N,N′-(2S,2′S)-1,1′-(dodecane-1,12- | N,N′-(2S,2′S)-1,1′-(tridecane-1,13- |
| diylbis(azanediyl))bis(3-methyl-1-oxobutane- | diylbis(azanediyl))bis(3-methyl-1-oxobutane- |
| 2,1-diyl)diisonicotinamide | 2,1-diyl)diisonicotinamide |
| N,N′-(2S,2′S)-1,1′-(tetradecane-1,14- | N,N′-(2S,2′S)-1,1′-(hexadecane-1,16- |
| diylbis(azanediyl))bis(3-methyl-1-oxobutane- | diylbis(azanediyl))bis(3-methyl-1-oxobutane- |
| 2,1-diyl)diisonicotinamide | 2,1-diyl)diisonicotinamide |
| N,N′-(2S,2′S)-1,1′-(octadecane-1,18- | |
| diylbis(azanediyl))bis(3-methyl-1-oxobutane- | |
| 2,1-diyl)diisonicotinamide | |

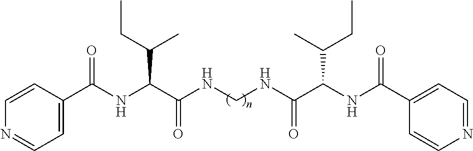

| | |
|---|---|
| N-[(1S)-2-methyl-1-[2-[[(2S)-3-methyl-2- | N-[(1S)-2-methyl-1-[3-[[(2S)-3-methyl-2- |
| (pyridine-4- | (pyridine-4- |
| carbonylamino)pentanoyl]amino]ethylcarbamoyl] | carbonylamino)pentanoyl]amino]propylcarbamoyl] |
| butyl]pyridine-4-carboxamide | butyl]pyridine-4-carboxamide |
| N-[(1S)-2-methyl-1-[4-[[(2S)-3-methyl-2- | N-[(1S)-2-methyl-1-[5-[[(2S)-3-methyl-2- |
| (pyridine-4- | (pyridine-4- |
| carbonylamino)pentanoyl]amino]butylcarbamoyl] | carbonylamino)pentanoyl]amino]pentylcarbamoyl] |
| butyl]pyridine-4-carboxamide | butyl]pyridine-4-carboxamide |
| N-[(1S)-2-methyl-1-[6-[[(2S)-3-methyl-2- | N-[(1S)-2-methyl-1-[7-[[(2S)-3-methyl-2- |
| (pyridine-4- | (pyridine-4- |
| carbonylamino)pentanoyl]amino]hexylcarbamoyl] | carbonylamino)pentanoyl]amino]heptylcarbamoyl] |
| butyl]pyridine-4-carboxamide | butyl]pyridine-4-carboxamide |
| N-[(1S)-2-methyl-1-[8-[[(2S)-3-methyl-2- | N-[(1S)-2-methyl-1-[9-[[(2S)-3-methyl-2- |
| (pyridine-4- | (pyridine-4- |
| carbonylamino)pentanoyl]amino]octylcarbamoyl] | carbonylamino)pentanoyl]amino]nonylcarbamoyl] |
| butyl]pyridine-4-carboxamide | butyl]pyridine-4-carboxamide |
| N-[(1S)-2-methyl-1-[10-[[(2S)-3-methyl-2- | N-[(1S)-2-methyl-1-[11-[[(2S)-3-methyl-2- |
| (pyridine-4- | (pyridine-4- |
| carbonylamino)pentanoyl]amino]decylcarbamoyl] | carbonylamino)pentanoyl]amino]undecylcarbamoyl] |
| butyl]pyridine-4-carboxamide | butyl]pyridine-4-carboxamide |
| N-[(1S)-2-methyl-1-[12-[[(2S)-3-methyl-2- | |
| (pyridine-4- | |
| carbonylamino)pentanoyl]amino]dodecylcarbamoyl] | |
| butyl]pyridine-4-carboxamide | |

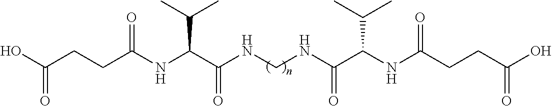

| | |
|---|---|
| | (6S,13S)-6,13-diisopropyl-4,7,12,15-tetraoxo- |
| | 5,8,11,14-tetraazaoctadecane-1,18-dioic acid |
| (6S,14S′)-6,14-diisopropyl-4,7,13,16-tetraoxo- | (6S,15S)-6,15-diisopropyl-4,7,14,17-tetraoxo- |
| 5,8,12,15-tetraazanonadecane-1,19-dioic acid | 5,8,13,16-tetraazaeicosane-1,20-dioic acid |
| (6S,16S)-6,16-diisopropyl-4,7,15,18-tetraoxo- | (6S,17S)-6,17-diisopropyl-4,7,16,19-tetraoxo- |
| 5,8,14,17-tetraazaheneicosane-1,21-dioic acid | 5,8,15,18-tetraazadocosane-1,22-dioic acid |
| (6S,18S)-6,18-diisopropyl-4,7,17,20-tetraoxo- | (6S,19S)-6,19-diisopropyl-4,7,18,21-tetraoxo- |
| 5,8,16,19-tetraazatricosane-1,23-dioic acid | 5,8,17,20-tetraazatetracosane-1,24-dioic acid |
| (6S,20S)-6,20-diisopropyl-4,7,19,22-tetraoxo- | (6S,21S)-6,21-diisopropyl-4,7,20,23-tetraoxo- |
| 5,8,18,21-tetraazapentacosane-1,25-dioic acid | 5,8,19,22-tetraazahexacosane-1,26-dioic acid |
| (6S,22S)-6,22-diisopropyl-4,7,21,24-tetraoxo- | (6S,23S)-6,23-diisopropyl-4,7,22,25-tetraoxo- |
| 5,8,20,23-tetraazaheptacosane-1,27-dioic acid | 5,8,21,24-tetraazaoctacosane-1,28-dioic acid |

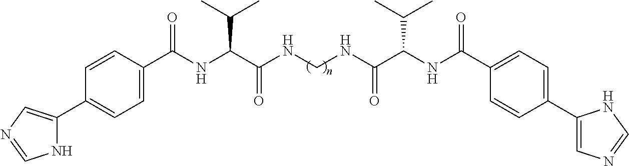

| | |
|---|---|
| N,N′-(2S,2′S)-1,1′-(ethane-1,2- | N,N′-(2S,2′S)-1,1′-(propane-1,3- |
| diylbis(azanediyl))bis(3-methyl-1-oxobutane- | diylbis(azanediyl))bis(3-methyl-1-oxobutane- |
| 2,1-diyl)bis(4-(1H-imidazol-5-yl)benzamide) | 2,1-diyl)bis(4-(1H-imidazol-5-yl)benzamide) |
| N,N′-(2S,2′S)-1,1′-(butane-1,4- | N,N′-(2S,2′S)-1,1′-(pentane-1,5- |
| diylbis(azanediyl))bis(3-methyl-1-oxobutane- | diylbis(azanediyl))bis(3-methyl-1-oxobutane- |
| 2,1-diyl)bis(4-(1H-imidazol-5-yl)benzamide) | 2,1-diyl)bis(4-(1H-imidazol-5-yl)benzamide) |
| N,N′-(2S,2′S)-1,1′-(hexane-1,6- | N,N′-(2S,2′S)-1,1′-(heptane-1,7- |
| diylbis(azanediyl))bis(3-methyl-1-oxobutane- | diylbis(azanediyl))bis(3-methyl-1-oxobutane- |
| 2,1-diyl)bis(4-(1H-imidazol-5-yl)benzamide) | 2,1-diyl)bis(4-(1H-imidazol-5-yl)benzamide) |
| N,N′-(2S,2′S)-1,1′-(octane-1,8- | N,N′-(2S,2′S)-1,1′-(nonane-1,9- |
| diylbis(azanediyl))bis(3-methyl-1-oxobutane- | diylbis(azanediyl))bis(3-methyl-1-oxobutane- |
| 2,1-diyl)bis(4-(1H-imidazol-5-yl)benzamide) | 2,1-diyl)bis(4-(1H-imidazol-5-yl)benzamide) |
| N,N′-(2S,2′S)-1,1′-(decane-1,10- | N,N′-(2S,2′S)-1,1′-(undecane-1,11- |
| diylbis(azanediyl))bis(3-methyl-1-oxobutane- | diylbis(azanediyl))bis(3-methyl-1-oxobutane- |
| 2,1-diyl)bis(4-(1H-imidazol-5-yl)benzamide) | 2,1-diyl)bis(4-(1H-imidazol-5-yl)benzamide) |
| N,N′-(2S,2′S)-1,1′-(dodecane-1,12- | |
| diylbis(azanediyl))bis(3-methyl-1-oxobutane- | |
| 2,1-diyl)bis(4-(1H-imidazol-5-yl)benzamide) | |

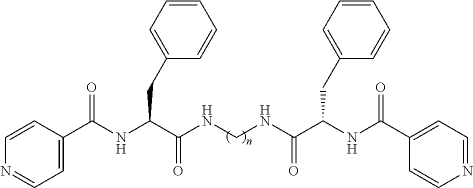

| | |
|---|---|
| | N,N′-(2S,2′S)-1,1′-(ethane-1,2- |
| | diylbis(azanediyl))bis(1-oxo-3- |
| | phenylpropane-2,1-diyl)diisonicotinamide |
| N,N′-(2S,2′S)-1,1′-(propane-1,3- | N,N′-(2S,2′S)-1,1′-(butane-1,4- |
| diylbis(azanediyl))bis(1-oxo-3- | diylbis(azanediyl))bis(1-oxo-3- |
| phenylpropane-2,1-diyl)diisonicotinamide | phenylpropane-2,1-diyl)diisonicotinamide |
| N,N′-(2S,2′S)-1,1′-(pentane-1,5- | N,N′-(2S,2′S)-1,1′-(hexane-1,6- |
| diylbis(azanediyl))bis(1-oxo-3- | diylbis(azanediyl))bis(1-oxo-3- |
| phenylpropane-2,1-diyl)diisonicotinamide | phenylpropane-2,1-diyl)diisonicotinamide |
| N,N′-(2S,2′S)-1,1′-(heptane-1,7- | N,N′-(2S,2′S)-1,1′-(octane-1,8- |
| diylbis(azanediyl))bis(1-oxo-3- | diylbis(azanediyl))bis(1-oxo-3- |
| phenylpropane-2,1-diyl)diisonicotinamide | phenylpropane-2,1-diyl)diisonicotinamide |
| N,N′-(2S,2′S)-1,1′-(nonane-1,9- | N,N′-(2S,2′S)-1,1′-(decane-1,10- |
| diylbis(azanediyl))bis(1-oxo-3- | diylbis(azanediyl))bis(1-oxo-3- |
| phenylpropane-2,1-diyl)diisonicotinamide | phenylpropane-2,1-diyl)diisonicotinamide |
| N,N′-(2S,2′S)-1,1′-(undecane-1,11- | N,N′-(2S,2′S)-1,1′-(dodecane-1,12- |
| diylbis(azanediyl))bis(1-oxo-3- | diylbis(azanediyl))bis(1-oxo-3- |
| phenylpropane-2,1-diyl)diisonicotinamide | phenylpropane-2,1-diyl)diisonicotinamide |
| N,N′-(2S,2′S)-1,1′-(tridecane-1,13- | N,N′-(2S,2′S)-1,1′-(tetradecane-1,14- |
| diylbis(azanediyl))bis(1-oxo-3- | diylbis(azanediyl))bis(1-oxo-3- |
| phenylpropane-2,1-diyl)diisonicotinamide | phenylpropane-2,1-diyl)diisonicotinamide |
| N,N′-(2S,2′S)-1,1′-(hexadecane-1,16- | N,N′-(2S,2′S)-1,1′-(octadecane-1,18- |
| diylbis(azanediyl))bis(1-oxo-3- | diylbis(azanediyl))bis(1-oxo-3- |
| phenylpropane-2,1-diyl)diisonicotinamide | phenylpropane-2,1-diyl)diisonicotinamide |

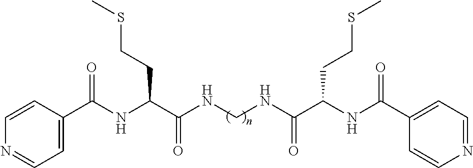

| | |
|---|---|
| N-[(1S)-3-methylsulfanyl-1-[2-[[(2S)-4- | N-[(1S)-3-methylsulfanyl-1-[3-[[(2S)-4- |
| methylsulfanyl-2-(pyridine-4- | methylsulfanyl-2-(pyridine-4- |
| carbonylamino)butanoyl]amino]ethylcarbamoyl] | carbonylamino)butanoyl]amino]propylcarbamoyl] |
| propyl]pyridine-4-carboxamide | propyl]pyridine-4-carboxamide |
| N-[(1S)-3-methylsulfanyl-1-[4-[[(2S)-4- | N-[(1S)-3-methylsulfanyl-1-[5-[[(2S)-4- |
| methylsulfanyl-2-(pyridine-4- | methylsulfanyl-2-(pyridine-4- |
| carbonylamino)butanoyl]amino]butylcarbamoyl] | carbonylamino)butanoyl]amino]pentylcarbamoyl] |
| propyl]pyridine-4-carboxamide | propyl]pyridine-4-carboxamide |
| N-[(1S)-3-methylsulfanyl-1-[6-[[(2S)-4- | N-[(1S)-3-methylsulfanyl-1-[7-[[(2S)-4- |
| methylsulfanyl-2-(pyridine-4- | methylsulfanyl-2-(pyridine-4- |
| carbonylamino)butanoyl]amino]hexylcarbamoyl] | carbonylamino)butanoyl]amino]heptylcarbamoyl] |
| propyl]pyridine-4-carboxamide | propyl]pyridine-4-carboxamide |
| N-[(1S)-3-methylsulfanyl-1-[8-[[(2S)-4- | N-[(1S)-3-methylsulfanyl-1-[9-[[(2S)-4- |
| methylsulfanyl-2-(pyridine-4- | methylsulfanyl-2-(pyridine-4- |
| carbonylamino)butanoyl]amino]octylcarbamoyl] | carbonylamino)butanoyl]amino]nonylcarbamoyl] |
| propyl]pyridine-4-carboxamide | propyl]pyridine-4-carboxamide |
| N-[(1S)-3-methylsulfanyl-1-[10-[[(2S)-4- | N-[(1S)-3-methylsulfanyl-1-[11-[[(2S)-4- |
| methylsulfanyl-2-(pyridine-4- | methylsulfanyl-2-(pyridine-4- |
| carbonylamino)butanoyl]amino]decylcarbamoyl] | carbonylamino)butanoyl]amino]undecylcarbamoyl] |
| propyl]pyridine-4-carboxamide | propyl]pyridine-4-carboxamide |
| N-[(1S)-3-methylsulfanyl-1-[12-[[(2S)-4- | |
| methylsulfanyl-2-(pyridine-4- | |
| carbonylamino)butanoyl]amino]dodecylcarbamoyl] | |
| propyl]pyridine-4-carboxamide | |

The more preferred di-amido gellants are selected from the group consisting of: N,N′-(2S,2′S)-1,1′-(ethane-1,2-diylbis(azanediyl))bis(3-methyl-1-oxobutane-2,1-diyl)diisonicotinamide, N,N′-(2S,2′S)-1,1′-(propane-1,3-diylbis(azanediyl))bis(3-methyl-1-oxobutane-2,1-diyl)diisonicotinamide, N,N′-(2S,2′S)-1,1′-(butane-1,4-diylbis(azanediyl))bis(3-methyl-1-oxobutane-2,1-diyl)diisonicotinamide, N,N′-(2S,2′S)-1,1′-(pentane-1,5-diylbis(azanediyl))bis(3-methyl-1-oxobutane-2,1-diyl)diisonicotinamide, N,N′-(2S,2′S)-1,1′-(hexane-1,6-diylbis(azanediyl))bis(3-methyl-1-oxobutane-2,1-diyl)diisonicotinamide, N,N′-(2S,2′S)-1,1′-(heptane-1,7-diylbis(azanediyl))bis(3-methyl-1-oxobutane-2,1-diyl)diisonicotinamide, N,N′-(2S,2′S)-1,1′-(octane-1,8-diylbis(azanediyl))bis(3-methyl-1-oxobutane-2,1-diyl)diisonicotinamide, N,N′-(2S,2′S)-1,1′-(nonane-1,9-diylbis(azanediyl))bis(3-methyl-1-oxobutane-2,1-diyl)diisonicotinamide, N,N′-(2S,2′S)-1,1′-(decane-1,10-diylbis(azanediyl))bis(3-methyl-1-oxobutane-2,1-diyl)diisonicotinamide, N,N′-(2S,2′S)-1,1′-(undecane-1,11-diylbis(azanediyl))bis(3-methyl-1-oxobutane-2,1-diyl)diisonicotinamide, N,N′-(2S,2′S)-1,1′-(dodecane-1,12-diylbis(azanediyl))bis(3-methyl-1-oxobutane-2,1-diyl)diisonicotinamide, N,N′-(2S,2′S)-1,1′-(tridecane-1,13-diylbis(azanediyl))bis(3-methyl-1-oxobutane-2,1-diyl)diisonicotinamide, N,N′-(2S,2′S)-1,1′-(tetradecane-1,14-diylbis(azanediyl))bis(3-methyl-1-oxobutane-2,1-diyl)diisonicotinamide, N,N′-(2S,2′S)-1,1′-(hexadecane-1,16-diylbis(azanediyl))bis(3-methyl-1-oxobutane-2,1-diyl)diisonicotinamide, N,N′-(2S,2′S)-1,1′-(octadecane-1,18-diylbis(azanediyl))bis(3-methyl-1-oxobutane-2,1-diyl)diisonicotinamide, N-[(1S)-2-methyl-1-[2-[[(2S)-3-methyl-2-(pyridine-4-carbonylamino)pentanoyl]amino]ethylcarbamoyl]butyl]pyridine-4-carboxamide, N-[(1S)-2-methyl-1-[4-[[(2S)-3-methyl-2-(pyridine-4-carbonylamino)pentanoyl]amino]butylcarbamoyl]butyl]pyridine-4-carboxamide, N-[(1S)-2-methyl-1-[6-[[(2S)-3-methyl-2-(pyridine-4-carbonylamino)pentanoyl]amino]hexylcarbamoyl]butyl]pyridine-4-carboxamide, N-[(1S)-2-methyl-1-[8-[[(2S)-3-methyl-2-(pyridine-4-carbonylamino)pentanoyl]amino]octylcarbamoyl]butyl]pyridine-4-carboxamide, N-[(1S)-2-methyl-1-[10-[[(2S)-3-methyl-2-(pyridine-4-carbonylamino)pentanoyl]amino]decylcarbamoyl]butyl]pyridine-4-carboxamide, N-[(1S)-2-methyl-1-[12-[[(2S)-3-methyl-2-(pyridine-4-carbonylamino)pentanoyl]amino]dodecylcarbamoyl]butyl]pyridine-4-carboxamide, N-[(1S)-2-methyl-1-[3-[[(2S)-3-methyl-2-(pyridine-4-carbonylamino)pentanoyl]amino]propylcarbamoyl]butyl]pyridine-4-carboxamide, N-[(1S)-2-methyl-1-[5-[[(2S)-3-methyl-2-(pyridine-4-carbonylamino)pentanoyl]amino]pentylcarbamoyl]butyl]pyridine-4-carboxamide, N-[(1S)-2-methyl-1-[7-[[(2S)-3-methyl-2-(pyridine-4-carbonylamino)pentanoyl]amino]heptylcarbamoyl]butyl]pyridine-4-carboxamide, N-[(1S)-2-methyl-1-[9-[[(2S)-3-methyl-2-(pyridine-4-carbonylamino)pentanoyl]amino]nonylcarbamoyl]butyl]pyridine-4-carboxamide, N-[(1S)-2-methyl-1-[11-[[(2S)-3-methyl-2-(pyridine-4-carbonylamino)pentanoyl]amino]undecylcarbamoyl]butyl]pyridine-4-carboxamide, N-[(1S)-3-methylsulfanyl-1-[2-[[(2S)-4-methylsulfanyl-2-(pyridine-4-carbonylamino)butanoyl]amino]ethylcarbamoyl]propyl]pyridine-4-carboxamide, N-[(1S)-3-methylsulfanyl-1-[3-[[(2S)-4-methylsulfanyl-2-(pyridine-4-carbonylamino)butanoyl]amino]propylcarbamoyl]propyl]pyridine-4-carboxamide, N-[(1S)-3-methylsulfanyl-1-[4-[[(2S)-4-methylsulfanyl-2-(pyridine-4-carbonylamino)butanoyl]amino]butylcarbamoyl]propyl]pyridine-4-carboxamide, N-[(1S)-3-methylsulfanyl-1-[5-[[(2S)-4-methylsulfanyl-2-(pyridine-4-carbonylamino)butanoyl]amino]pentylcarbamoyl]propyl]pyridine-4-carboxamide, N-[(1S)-3-methylsulfanyl-1-[6-[[(2S)-4-methylsulfanyl-2-(pyridine-4-carbonylamino)butanoyl]amino]hexylcarbamoyl]propyl]pyridine-4-carboxamide, N-[(1S)-3-methylsulfanyl-1-[7-[[(2S)-4-methylsulfanyl-2-(pyridine-4-carbonylamino)butanoyl]amino]heptylcarbamoyl]propyl]pyridine-4-carboxamide, N-[(1S)-3-methylsulfanyl-1-[8-[[(2S)-4-methylsulfanyl-2-(pyridine-4-carbonylamino)butanoyl]amino]octylcarbamoyl]propyl]pyridine-4-carboxamide, N-[(1S)-3-methylsulfanyl-1-[9-[[(2S)-4-methylsulfanyl-2-(pyridine-4-carbonylamino)butanoyl]amino]nonylcarbamoyl]propyl]pyridine-4-carboxamide, N-[(1S)-3-methylsulfanyl-1-[10-[[(2S)-4-methylsulfanyl-2-(pyridine-4-carbonylamino)butanoyl]amino]decylcarbamoyl]propyl]pyridine-4-carboxamide, N-[(1S)-3-methylsulfanyl-1-[11-[[(2S)-4-methylsulfanyl-2-(pyridine-4-carbonylamino)butanoyl]amino]undecylcarbamoyl]propyl]pyridine-4-carboxamide, N-[(1S)-3-methylsulfanyl-1-[12-[[(2S)-4-methylsulfanyl-2-(pyridine-4-carbonylamino)butanoyl]amino]dodecylcarbamoyl]propyl]pyridine-4-carboxamide, dibenzyl (2S,2′S)-1,1′-(ethane-1,2-diylbis(azanediyl))bis(3-methyl-1-oxobutane-2,1-diyl)dicarbamate, dibenzyl (2S,2′S)-1,1′-(butane-1,4-diylbis(azanediyl))bis(3-methyl-1-oxobutane-2,1-diyl)dicarbamate, dibenzyl (2S,2′S)-1,1′-(hexane-1,6-diylbis(azanediyl))bis(3-methyl-1-oxobutane-2,1-diyl)dicarbamate, dibenzyl (2S,2′S)-1,1′-(octane-1,8-diylbis(azanediyl))bis(3-methyl-1-oxobutane-2,1-diyl)dicarbamate, dibenzyl (2S,2′S)-1,1′-(decane-1,10-diylbis(azanediyl))bis(3-methyl-1-oxobutane-2,1-diyl)dicarbamate, dibenzyl (2S,2′S)-1,1′-(dodecane-1,12-diylbis(azanediyl))bis(3-methyl-1-oxobutane-2,1-diyl)dicarbamate, dibenzyl (2S,2′S)-1,1′-(propane-1,3-diylbis(azanediyl))bis(3-methyl-1-oxobutane-2,1-diyl)dicarbamate, dibenzyl (2S,2′S)-1,1′-(pentane-1,5-diylbis(azanediyl))bis(3-methyl-1-oxobutane-2,1-diyl)dicarbamate, dibenzyl (2S,2′S)-1,1′-(heptane-1,7-diylbis(azanediyl))bis(3-methyl-1-oxobutane-2,1-diyl)dicarbamate, dibenzyl (2S,2′S)-1,1′-(nonane-1,9-diylbis(azanediyl))bis(3-methyl-1-oxobutane-2,1-diyl)dicarbamate, dibenzyl (2S,2′S)-1,1′-(undecane-1,11-diylbis(azanediyl))bis(3-methyl-1-oxobutane-2,1-diyl)dicarbamate, and mixtures thereof.

The most preferred di-amido gellants are selected from the group consisting of: N,N′-(2S,2′S)-1,1′-(propane-1,3-diylbis(azanediyl))bis(3-methyl-1-oxobutane-2,1-diyl)diisonicotinamide, N,N′-(2S,2′S)-1,1′-(dodecane-1,12-diylbis(azanediyl))bis(3-methyl-1-oxobutane-2,1-diyl)diisonicotinamide, N,N′-(2S,2′S)-1,1′-(tridecane-1,13-diylbis(azanediyl))bis(3-methyl-1-oxobutane-2,1-diyl)diisonicotinamide, N-[(1S)-2-methyl-1-[12-[[(2S)-3-methyl-2-(pyridine-4-carbonylamino)pentanoyl]amino]dodecylcarbamoyl]butyl]pyridine-4-carboxamide, N-[(1S)-2-methyl-1-[3-[[(2S)-3-methyl-2-(pyridine-4-carbonylamino)pentanoyl]amino]propylcarbamoyl]butyl]pyridine-4-carboxamide, N-[(1S)-3-methylsulfanyl-1-[3-[[(2S)-4-methylsulfanyl-2-(pyridine-4-carbonylamino)butanoyl]amino]propylcarbamoyl]propyl]pyridine-4-carboxamide, N-[(1S)-3-methylsulfanyl-1-[12-[[(2S)-4-methylsulfanyl-2-(pyridine-4-carbonylamino)butanoyl]amino]dodecylcarbamoyl]propyl]pyridine-4-carboxamide, dibenzyl (2S,2′S)-1,1′-(dodecane-1,12-diylbis(azanediyl))bis(3-methyl-1-oxobutane-2,1-diyl)dicarbamate, dibenzyl (2S,2′S)-1,1′-(propane-1,3-diylbis(azanediyl))bis(3-methyl-1-oxobutane-2,1-diyl)dicarbamate, and mixtures thereof.

### Adjunct Ingredients:

The fluid composition of the unit dose articles of the present invention may also include conventional detergent ingredients selected from the group consisting of: surfactants, enzymes, enzyme stabilizers, amphiphilic alkoxylated grease cleaning polymers, clay soil cleaning polymers, soil release polymers, soil suspending polymers, bleaching systems, optical brighteners, hueing dyes, particulate material, perfume and other odour control agents including perfume delivery systems, hydrotropes, suds suppressors, fabric care benefit agents, pH adjusting agents, dye transfer inhibiting agents, preservatives, non-fabric substantive dyes and mixtures thereof. Some of the optional ingredients which can be used are described in greater detail as follows:

Fluid compositions of the unit dose articles of the present invention may comprise a surfactant. When present, the surfactant is present at a level of from 1% to 70%, preferably from 5% to 60% by weight, more preferably from 10% to 50%, and most preferably from 15% to 45% by weight of the fluid composition. The surfactant is preferably selected from the group: anionic, nonionic surfactants and mixtures thereof. The preferred ratio of anionic to nonionic surfactant is from 100:0 (i.e. no nonionic surfactant) to 5:95, more preferably from 99:1 to 1:4, most preferably 5:1 to 1.5:1, particularly for water soluble laundry detergent articles.

The fluid compositions of the present invention preferably comprises from 1 to 50%, more preferably from 5 to 40%, most preferably from 10 to 30% by weight of one or more anionic surfactants. Preferred anionic surfactant are selected from the group consisting of: C11-C18 alkyl benzene sulphonates, C10-C20 linear or branched-chain or random alkyl sulphates, C10-C18 alkyl ethoxy sulphates, C5-C22 mid-chain branched alkyl sulphates, mid-chain branched alkyl alkoxy sulphates, C10-C18 alkyl alkoxy carboxylates comprising 1-5 ethoxy units, modified alkylbenzene sulphonate, C12-C20 methyl ester sulphonate, C10-C18 alpha-olefin sulphonate, C6-C20 sulphosuccinates, and mixtures thereof. The compositions of the present invention comprise preferably at least one sulphonic acid surfactant, such as a linear alkyl benzene sulphonic acid, or the water-soluble salt forms. When mixtures are used, a suitable average total number of carbon atoms for the alkyl moieties is preferably within the range of from 14.5 to 17.5. The anionic surfactants are typically present in the form of their salts with alkanolamines or alkali metals such as sodium and potassium.

The fluid compositions of the unit dose articles of the present invention preferably comprise up to 30%, more preferably from 1 to 15%, most preferably from 2 to 10% by weight of one or more nonionic surfactants. Suitable nonionic surfactants include, but are not limited to C12-C18 alkyl ethoxylates (“AE”) including the so-called narrow peaked alkyl ethoxylates, C6-C12 alkyl phenol alkoxylates (especially ethoxylates and mixed ethoxy/propoxy), block alkylene oxide condensate of C6-C12 alkyl phenols, alkylene oxide condensates of C8-C22 alkanols and ethylene oxide/propylene oxide block polymers (Pluronic®-BASF Corp.), as well as semi polar nonionics (e.g., amine oxides and phosphine oxides). An extensive disclosure of suitable nonionic surfactants can be found in U.S. Pat. No. 3,929,678.

The fluid compositions of the unit dose articles of the present invention may comprise additional surfactant selected from the group consisting: cationic, amphoteric and/or zwitterionic surfactants, and mixtures thereof. Examples include alkyltrimethylammonium salts, such as C12 alkyltrimethylammonium chloride, or their hydroxyalkyl substituted analogs. The fluid compositions may comprise 1% or more of cationic surfactant. Suitable amphoteric surfactants for use in the present invention include, but are not limited to: cocoamphoacetate, cocoamphodiacetate, lauroamphoacetate, lauroamphodiacetate, and mixtures thereof. Zwitterionics such as betaines are suitable for this invention.

Furthermore, amine oxide surfactants having the formula: R(EO)ₓ(PO)_{y}(BO)_{z}N(O)(CH₂R)₂.qH₂O (I) are also useful in fluid compositions. R is a relatively long-chain hydrocarbyl moiety which can be saturated or unsaturated, linear or branched, and can contain from 8 to 20, preferably from 10 to 16 carbon atoms, and is more preferably C12-C16 primary alkyl. R′ is a short-chain moiety preferably selected from hydrogen, methyl and —CH₂OH. When x+y+z is different from 0, EO is ethyleneoxy, PO is propyleneneoxy and BO is butyleneoxy. Amine oxide surfactants are illustrated by C12-C14 alkyldimethyl amine oxide.

Non-aqueous compositions that comprise enzymes typically require little or no enzyme inhibitors, since the low water levels typically render the enzyme inactive. At higher water levels, enzyme activity increases, leading to a shorter enzyme life and incompatibility with other ingredients. Since the di-amido gellants of the present invention are able to sequester much of the free water, they are able to inhibit the enzymes, and hence improve enzyme stability in fluid compositions that comprise water.

The fluid compositions of the unit dose articles of the present invention may comprise from 0.0001% to 8% by weight of detersive enzyme which provide improved cleaning performance and/or fabric care benefits. Such fluid compositions have a neat pH of from 6 to 10.5. Suitable enzymes can be selected from the group consisting of: lipase, protease, cellulase, amylase, mannanase, pectate lyase, xyloglucanase, and mixtures thereof. A preferred enzyme combination comprises a cocktail of conventional detersive enzymes such as lipase, protease, and amylase. When a protease enzyme is present, the protease is preferably inhibited. The protease may be inhibited by the relatively low water content of the fluid composition, or by the addition of a suitable inhibitor. Alternatively, the enzyme combination does not include proteases. Enzymes, particularly protease and lipase, may be encapsulated.

If necessary, suitable protease inhibitors, particularly for the inhibition of serine proteases, include derivates of boronic acid, especially phenyl boronic acid and its derivatives, and peptide aldehydes, including tripeptide aldehydes. Examples of such compounds are disclosed in WO 98/13458 A1, WO 07/113241 A1, and U.S. Pat. No. 5,972,873. Suitable protease inhibitors may comprise 4-formyl phenyl boronic acid.

Preferably, the fluid composition comprises from 0.1% to 7%, more preferably from 0.2% to 3%, of a polymer deposition aid. As used herein, “polymer deposition aid” refers to any cationic polymer or combination of cationic polymers that significantly enhance deposition of a fabric care benefit agent onto substrates (such as fabric) during washing (such as laundering). Suitable polymer deposition aids can comprise a cationic polysaccharide and/or a copolymer.

The detergent compositions herein may optionally contain from 0.01 to 10% by weight of one or more cleaning polymers that provide for broad-range soil cleaning of surfaces and fabrics and/or suspension of the soils. Any suitable cleaning polymer may be of use. Useful cleaning polymers are described in U.S. 2009/0124528A1. Non-limiting examples of useful categories of cleaning polymers include: amphiphilic alkoxylated grease cleaning polymers; clay soil cleaning polymers; soil release polyers; and soil suspending polymers.

One embodiment is a unit dose articles comprising a fluid composition, wherein the fluid composition is a fluid laundry bleach additive comprising from 0.1% to 12% by weight of a bleach active or bleach system, preferably a peroxide bleach, and further comprises a neat pH of from 2 to 6. Another embodiment is a unit dose article comprising a fluid laundry detergent composition comprising: from 0.1% to 12% by weight of the bleach, and a neat pH of from 6.5 to 10.5, provided that if the fluid composition comprises an enzyme, the bleach active is preferably at least partially physically separated, more preferably fully separated, from the enzyme.

Suitable bleach actives include hydrogen peroxide sources, such as hydrogen peroxide itself; perborates, e.g., sodium perborate (any hydrate but preferably the mono- or tetra-hydrate); sodium carbonate peroxyhydrate or equivalent percarbonate salts; sodium pyrophosphate peroxyhydrate, urea peroxyhydrate, persulfates, sodium peroxide, and mixtures thereof. Sodium perborate monohydrate and sodium percarbonate are particularly preferred.

The bleaching systems of use in the present invention may also include ingredients selected from the group consisting of: bleach activators, hydrogen peroxide, hydrogen peroxide sources, organic peroxides, metal-containing bleach catalysts, transition metal complexes of macropolycyclic rigid ligands, organic bleach catalysts, preformed peracids, photobleaches and mixtures thereof. The preferred preformed peracid is Phthalimido peroxycaproic acid (PAP).

For improved stability before use, the bleach active is preferably at least partially physically separated, more preferably fully separated, from ingredients that are sensitive to the bleach active, such as enzymes. In one embodiment, the bleach active is a solid. In such embodiments, interaction between the bleach active and bleach sensitive ingredients is inhibited by the solid-liquid phase boundary. In another embodiment, the bleach active is encapsulated by a water-soluble barrier which keeps the majority of the bleach active isolated from bleach sensitive ingredients. In yet another embodiment, the bleach active is in a different compartment from the bleach sensitive ingredients.

The fluid compositions comprised in unit dose articles of the present invention may include perfume delivery systems that enhance the deposition and release of perfume ingredients from treated substrate. Since such ingredients are typically supplied as aqueous suspensions or emulsions that comprise from 50% to 95%, more preferably from 60% to 85% water, they are particularly suited for unit dose articles of the present invention. Perfume delivery systems, methods of making certain perfume delivery systems and the uses of such perfume delivery systems are disclosed in U.S. 2007/0275866 A1, U.S. 2004/0110648 A1, U.S. 2004/0092414 A1, 2004/0091445 A1, 2004/0087476 A1, U.S. Pat. Nos. 6,531,444, 6,024,943, 6,042,792, 6,051,540, 4,540,721, and 4,973,422.

When used, the fluid composition preferably comprises from 0.001% to 20%, more preferably from 0.01% to 10%, even more preferably from 0.05% to 5%, most preferably from 0.1% to 0.5% by weight of the perfume delivery system. Preferred perfume delivery systems can be selected from the group consisting of: perfume microcapsules, pro-perfumes, polymer particles, functionalized silicones, and mixtures thereof.

If present, the perfume microcapsule wall material is typically selected from the group consisting of: melamine crosslinked with formaldehyde, melamine-dimethoxyethanol crosslinked with formaldehyde, polyacrylamide, silica, polyurea, polystyrene cross linked with divinylbenzene, polyurethane, polyacrylate based materials, polyacrylate formed from metthylmethacrylate/dimethylaminomethyl methacrylate, polyacrylate formed from amine acrylate and/or methacrylate and a strong acid, polyacrylate formed from a carboxylic acid acrylate and/or methacrylate monomer and a strong base; polyacrylate formed from an amine acrylate and/or methacrylate monomer and a carboxylic acid acrylate and/or carboxylic acid methacrylate monomer, silicone, urea crosslinked with formaldehyde or urea crosslinked with gluteraldehyde, gelatin, polyacrylates, acrylate monomers, and combinations thereof. Pro-perfumes are the result of a chemical reaction between one or more perfume raw materials and a carrier molecule, resulting in a covalent bond between the perfume raw material and the carrier material, which then dissociates after exposure to suitable triggers such as: moisture, enzymes, heat, light, pH change, autoxidation, a shift of the chemical equilibrium, a change in concentration or ionic strength, and mixtures thereof. Perfume ingredients can also be dissolved or dispersed into or onto a polymer particle, typically of sizes in the nanometer or micrometer range. Suitable functionalized silicones include amine-functionalized silicones.

The fluid composition of the unit dose articles of the present invention may further comprise: optical brighteners, hueing dyes, clays, mica, suds suppressors, perfume and odour control agents, and additional structurants. Non-limiting examples of suitable additional structurants can be selected from the group consisting of: di-benzylidene polyol acetal derivatives, bacterial cellulose, coated bacterial cellulose, non-polymeric crystalline hydroxyl-functional materials, polymeric structuring agents, and mixtures thereof. Mica is particularly suitable for compositions of the present invention, since mica is typically added as an as aqueous suspensions or emulsions that comprise from 50% to 95%, more preferably from 60% to 85% water.

### Process of Making:

The present invention also provides for a preferred process of making a unit dose article comprising the steps of:
(a) making a di-amido gellant premix comprising a di-amido gellant and a solvent;
(b) combining the di-amido gellant premix with a fluid feed, wherein the fluid feed comprises from 10% to 70% by weight water, to form a fluid composition; and
(c) encapsulating the fluid composition in a water soluble film.

Suitable solvents include water, non-aminofunctional solvents, and mixtures thereof. The fluid feed comprises some or all of the remaining ingredients of the fluid composition, in addition to water. The di-amido gellant premix, the fluid feed, and mixtures thereof, may include an anionic surfactant. The anionic surfactant may be incorporated in an acid form, such as linear alkylbenzene sulphonic acid. Alternatively, the anionic surfactant may be incorporated in a neutralised form, for instance neutralized by an alkali metal salt such as sodium hydroxide, or an alkanolamine such as monothenanolamine or triethanolamine. If present, the anionic surfactants used in steps (a) and (b) can be the same or different. The di-amido gellant premix, the detergent feed, and mixtures thereof may also include additional surfactants, such as a nonionic surfactant. A secondary structurant can be present in either the fluid feed, or the di-amido gellant premix.

The di-amido gellant premix can comprise less than 10%, preferably less than 5%, more preferably less than 2% by weight of water. Alternatively, the di-amido gellant premix can be free of water. In one embodiment, the di-amido gellant premix comprises a solvent, preferably an organic solvent, to solubilise the di-amido gellant.

In another embodiment, the process comprises the additional step of cooling the fluid composition. In yet another embodiment, the process comprises the additional step of adding heat sensitive ingredients such as detersive enzymes when the step of cooling the composition brings the compositional temperature below the temperature where the heat sensitive ingredients are subject to decomposition.

In one embodiment, the step of forming the di-amido gellant premix is performed at a temperature above which the said di-amido gellant dissolves in the solvent (for instance above 80° C., alternatively above 95° C.). Preferably, the temperature at which the premix is formed is at least 5° C., more preferably at least 10° C. higher than the temperature at which all the di-amido gellant is fully dissolved in the di-amido gellant premix.

In another embodiment, the step of combining the di-amido gellant premix with the fluid feed is performed by adding the di-amido gellant premix at a temperature of at least 80° C., to a fluid feed that is heated up to a temperature of not more than 60° C., preferably not more than 50° C. The heat-sensitive ingredients, such as enzymes, perfumes, bleach catalysts, photobleaches, bleaches and dyes are preferably added to the detergent feed after the di-amido gellant premix has been added, and after the temperature of the fluid composition is below 45° C., preferably below 30° C.

When the fluid detergent composition of the unit dose article comprises a pH-tuneable di-amido gellant, in step (a) of the preferred process, the di-amido gellant is a pH tuneable di-amido gellant, and the di-amido gellant premix is preferably at a pH such that the pH tuneable di-amido gellant is in an ionic form, that is non-viscosity building. Such processes typically include a step of adjusting the pH of the fluid composition, either during or after the addition of the di-amido gellant premix, such that the fluid composition is altered to a pH at which the pH tuneable di-amido gellant is at least partially nonionic, and builds viscosity.

Since di-amido gellant premixes comprising a pH tuneable di-amido gellant, and fluid compositions formed with such premixes, can be processed at temperatures of less than 50° C., preferably less than 30° C., di-amido gellant premixes comprising a pH tuneable di-amido gellant are particularly suitable for making fluid compositions that further comprise temperature-sensitive ingredients such as enzymes or perfumes.

Regardless of whether a pH tuneable di-amido gellant is used or not, the process may include a further step of adjusting the pH of the fluid composition before it is encapsulated in the water soluble film. The pH can be adjusted through the addition of a suitable acid or alkali. Suitable acids include linear alkylbenzene sulphonic acid (HLAS), chlorhydric acid, citric acid, sulphuric acid, lactic acid, nitric acid, oxalic acid, and mixtures thereof. Suitable alkalis include sodium hydroxide, potassium hydroxide, magnesium hydroxide, barium hydroxide, sodium carbonate, potassium carbonate, monoethanolamine, caesium hydroxide, strontium hydroxide, and mixtures thereof.

The fluid composition may be encapsulated in a water soluble film by any suitable means. For instance, the water-soluble film can be cut to an appropriate size, and then folded to form the necessary number and size of compartments. The edges can then be sealed using any suitable technology, for example heat sealing, wet sealing or pressure sealing. Preferably, a sealing source is brought into contact with said film, and heat or pressure is applied to seal the film material.

The water soluble film is typically introduced to a mould and a vacuum applied so that said film is flush with the inner surface of the mould, thus forming an indent or niche in said film material. This is referred to as vacuum-forming. Another suitable method is thermo-forming. Thermo-forming typically involves the step of forming a water-soluble film in a mould under application of heat, which allows said film to deform and take on the shape of the mould. Preferably, a combination of thermoforming and vacuum forming is used.

Typically more than one piece of water-soluble film material is used for making the unit dose article. For example, a first piece of film material can be heated and then vacuum pulled into the mould so that said first piece of film material is flush with the inner walls of the mould. The fluid composition is then introduced into the mould. A second piece of film material can then be positioned such that it completely overlaps with the first piece of film material. The first piece of film material and second piece of film material are sealed together. The first and second pieces of water-soluble film can be made of the same material or can be different materials.

In a process for preparing a multi-compartment unit dose article, a piece of water-soluble film material is folded at least twice, or at least three pieces of film material are used, or at least two pieces of film material are used wherein at least one piece of film material is folded at least once. The third piece of film material, or a folded piece of film material, creates a barrier layer that, when the film materials are sealed together, divides the internal volume of the unit dose article into two or more compartments.

A multi-compartment unit dose article may also be prepared by fitting a first piece of film material into a mould. A composition, or component thereof, can then be poured into the mould. A pre-formed compartment can then be placed over the mould containing the composition, or component thereof. The pre-formed compartment also preferably contains a composition, or component thereof. The pre-formed compartment and said first piece of water-soluble film material are sealed together to form the multi-compartment unit dose article.

### Test Methods:

1. pH Measurement:
   The pH is measured on the neat composition, at 25° C., using a Santarius PT-10P pH meter with gel-filled probe (such as the Toledo probe, part number 52 000 100), calibrated according to the instructions manual.
2. Minimum gelling concentration (MGC)
   MGC is calculated by a tube inversion method based on R. G. Weiss, P. Terech; “Molecular Gels: Materials with self-assembled fibrillar structures” 2006 springer, p 243. In order to determine the MGC, three screenings are done:
      a) First screening: prepare several vials increasing the di-amido gellant concentration from 0.5% to 5.0 weight % in 0.5% steps
      b) Determine in which interval the gel is formed (one inverted sample still flowing and the next one is already a strong gel). In case no gel is formed at 5%, higher concentrations are used.
      c) Second screening: prepare several vials increasing the di-amido gellant concentration in 0.1 weight % steps in the interval determined in the first screening.
      d) Determine in which interval the gel is formed (one inverted sample still flowing and the next one is already a strong gel)
      e) Third screening: in order to have a very precise percentage of the MGC, run a third screening in 0.025 weight % steps in the interval determined in the second screening.
      f) The Minimum Gelling Concentration (MGC) is the lowest concentration which forms a gel in the third screening (does not flow on inversion of the sample).

For each screening, samples are prepared and treated as follows: 8 mL vials (Borosilacate glass with Teflon cap, ref. B7857D, Fisher Scientific Bioblock) are filled with 2.0000±0.0005 g (KERN ALJ 120-4 analytical balance with ±0.1 mg precision) of the fluid (comprising the fluid composition and di-amido gellant) for which we want to determine the MGC. The vial is sealed with the screw cap and left for 10 minutes in an ultrasound bath (Elma Transsonic T 710 DH, 40 kHz, 9.5 L, at 25° C. and operating at 100% power) in order to disperse the solid in the fluid. Complete dissolution is then achieved by heating, using a heating gun (Bosch PHG-2), and gentle mechanical stirring of the vials. It is crucial to observe a completely clear solution. Handle vials with care. While they are manufactured to resist high temperatures, a high solvent pressure may cause the vials to explode. Vials are cooled to 25° C., for 10 min in a thermostatic bath (Compatible Control Thermostats with controller CC2, D77656, Huber). Vials are inverted, left inverted for 1 minute, and then observed for which samples do not flow. After the third screening, the concentration of the sample that does not flow after this time is the MGC. For those skilled in the art, it is obvious that during heating solvent vapours may be formed, and upon cooling down the samples, these vapours can condense on top of the gel. When the vial is inverted, this condensed vapour will flow. This is discounted during the observation period. If no gels are obtained in the concentration interval, higher concentrations must be evaluated.
3. Rheology
   An AR-G2 rheometer from TA Instruments is used for rheological measurements.
   Plate: 40 mm standard steel parallel plate, 300 μm gap, at 20° C.
      Resting (low stress) viscosity: The resting (low stress) viscosity is determined under a constant stress of 0.1 Pa during a viscosity creep experiment over a 5 minute interval. Rheology measurements over the 5 minute interval are made after the composition has rested at zero shear rate for at least 10 minutes, between loading the sample in the rheometer and running the test. The data over the last 3 minutes are used to fit a straight line, and from the slope of this line, the low stress viscosity is calculated.
4. Method of measuring the solubility of water-soluble films

5.0 grams±0.1 gram of the water-soluble film is added in a pre-weighed 400 ml beaker and 245 ml±1 ml of distilled water at 10° C. is added. This is stirred vigorously on a magnetic stirrer set at 600 rpm, for 30 minutes. Then, the mixture is filtered through a sintered-glass filter with a pore size of maximum 20 microns. The water is dried off from the collected filtrate by any conventional method, and the weight of the remaining material is determined (which is the dissolved or dispersed fraction). Then, the percentage solubility or dispersibility can be calculated.
5. Method of measuring the dissolution time of water-soluble films

The film is cut and mounted into a folding frame slide mount for 24 mm by 36 mm diapositive film, without glass (part number 94.000.07, supplied by Else, The Netherlands, however plastic folding frames from other suppliers may be used).

A standard 600 ml glass beaker is filled with 500 ml of city water at 10° C. and agitated using a magnetic stirring rod such that the bottom of the vortex is at the height of the 400 ml graduation mark on the beaker.

The slide mount is clipped to a vertical bar and suspended into the water, with the 36 mm side horizontal, along the diameter of the beaker, such that the edge of the slide mount is 5 mm from the beaker side, and the top of the slide mount is at the height of the 400 ml graduation mark. The stop watch is started immediately the slide mount is placed in the water, and stopped when the film fully dissolves. This time is recorded as the “film dissolution time”.
6. The Wash-Residue Test:
   The wash residue test qualitatively measures the residual polymer after the water-soluble polymer is subjected to a cold water wash cycle.

For single compartment unit dose articles, 0.7 g of a 76 μm thick piece of the desired PVOH film is thermoformed to make a unit dose article measuring about 60×60 mm, which is filled with 37.5 ml of the desired fluid composition.

For three component unit dose articles, 0.6 g of a 76 μm thick piece of the desired PVOH film is thermoformed to make the three component unit dose article, measuring about 44×44 mm, which is filled with 17.5 ml of the fluid composition of the first compartment and 1.5 ml of the desired fluid composition of each of the second and third compartments. The sealed packet is then secured within a black velvet bag (23.5 cm×47 cm of 72% Cotton/28% black velvet, preferably Modal black velvet supplied by EQUEST U.K., and produced by DENHOLME VELVETS, Halifax Road, Denholme, Bradford, West Yorkshire, England) by stitching along the whole length of the bag opening side with a plastic thread.

The sealed velvet bag is then placed at the bottom of a washing machine drum (preferably a MIELE washing machine type W467 connected to a water temperature control system). To overcome machine-to-machine variation, preferably four machines should be used in each test with four samples of water-soluble polymer each secured within a velvet bag in each machine. The bags should be placed side-to-side in the bottom of the machine with different relative positions within each machine to avoid any effect of the positioning of the bag in the machine. The washing cycle is then engaged on a “wool cycle/cold” setting with a starting water temperature of 5° C.±1° C. (controlled by a water temperature control system) without any additional ballast load. At end of the washing cycle, the bag should be removed from the machine, opened and graded within fifteen minutes.

Grading is made by visual observation of the residue remaining in/on the bag after the wash. The qualitative scale is 0 (no residues) to 7 (the whole of the polymer film remains in the bag):
Grade 0: No residues
Grade 1: Maximum of 3 small spread spots of max. 2 cm diameter each, spots are flat a transparent
Grade 2: More than 3 small spots of 2 cm diameter each up to full Black unit dose article is covered with flat transparent film
Grade 2.5: Small opaque residue (soft PVOH) less than 1 cm diameter.
Grade 3: Opaque residue with a diameter between 1 and 2 cm (concentrated PVOH film)
Grade 4: Opaque residue with diameter between 3 and 4 cm diameter (concentrated PVOH film)
Grade 5: Thick residue with diameter between 4-6 cm diameter (+/−half of the unit dose article is not dissolved)
Grade 6: Lump of concentrated soft PVOH residue with a diameter <6 cm, more than half the unit dose article was not dissolved.
Grade 7: Full unit dose article not dissolved, PVOH is soft
   A water-soluble polymer film passes the Wash-Residue Test if the average residue grading for the sixteen trials is less than 4.5, preferably less than 3.
7. Water condensation test:

The water condensation test provides a gauge of the unit dose article stability in a package. 0.7 grams of a 76 μm thick PVOH film is thermoformed to a single compartment unit dose article, measuring about 60×60 mm, and the unit dose article is filled with 36 ml of the fluid composition. For evaluating multi-compartment unit dose articles, 0.6 grams of a 76 μm thick PVOH film is thermoformed into a three component unit dose article, measuring about 44×44 mm, that is filled with 17.5 ml of the fluid composition of the first compartment and 1.5 ml of each of the fluid composition of the second and third compartments. Then, the unit dose article is sealed into a plastic container of 10.5×7.5×5 cm and stored at 35° C. for 30 days, noting water condensation after 3, 15 and 30 days. If there is water condensation, unit dose articles containing that fluid composition would stick to each other in the package.

### EXAMPLES

The unit dose articles of comparative example 1, and example 1 of the present invention, were prepared as follows: A first section of water-soluble film (M8779, supplied by Monosol of Merrillville, Ind., US) was thermoformed into a mould having 25 compartments, before 36 mL of the fluid composition was added into each compartment. A second section of the water-soluble film (M8779) was then placed over the compartments so that it completely overlapped the first section of water-soluble film, and the two sections of water-soluble film sealed together. The sealed parts of the film were then cut to form the 25 individual unit dose articles:

| | Comparative | |
|---|---|---|
| | Example 1 | Example 1 |
| Ingredients | Weight % | |
| Sodium Diethylene Triamine Penta acetate | 0.5 | 0.5 |
| C₁₁₋₁₆ Alkylbenzene sulfonic acid | 7.6 | 7.6 |
| C₁₂₋₁₄ alkyl 9-ethoxylate | 6.2 | 6.2 |
| Citric acid | 0.5 | 05 |
| C₁₂₋₁₈ Fatty acid | 1.4 | 1.4 |
| ethylene diamine disuccinic acid | 3.5 | 3.5 |
| Calcium formate | 0.3 | 0.3 |
| Amphiphilic alkoxylated grease | 3.0 | 3.0 |
| cleaning polymer¹ | | |
| Chlorhydric acid | 0.8 | 0.8 |
| N,N′-(2S,2′S)-1,1′-(dodecane-1,12- | — | 0.78 |
| diylbis(azanediyl))bis(3-methyl-1- | | |
| oxobutane-2,1-diyl)diisonicotinamide | | |
| Water | 50 | 50 |
| Minors (antioxidant, sulfite, aesthetics, . . . ) | 0.5 | 0.5 |
| Buffers (monoethanolamine) | Up to pH 8 | Up to pH 8 |
| Solvents (1,2-propanediol, ethanol, . . . ) | Up to 100 | Up to 100 |

¹PG617 or PG640 (supplied by BASF, Germany)

Of the 25 unit dose articles of comparative example 1 that were made, 24 leaked due to film tearing or seal failure during making. The remaining unit dose article leaked after less than 1 hour storage at 35° C. In contrast, all of the unit dose articles of example 1 of the present invention survived both making, and storage at 35° C. for an hour. Thus, it is clear that robust, stable unit-dose articles can be formed, containing as high as 50 wt % water, when a di-amido gellant included in the fluid composition.

The unit dose articles of comparative example 2, and example 2 of the present invention, were prepared using the method of comparative example 1, and example 1 of the present invention.

| | Comparative | |
|---|---|---|
| | Example 2 | Example 2 |
| Ingredients | Weight % | |
| Sodium Diethylene Triamine Penta acetate | 0.6 | 0.6 |
| C₁₁₋₁₆ Alkylbenzene sulfonic acid | 9.1 | 9.1 |
| C₁₂₋₁₄ alkyl 9-ethoxylate | 7.5 | 7.5 |
| Citric acid | 0.6 | 0.6 |
| C₁₂₋₁₈ Fatty acid | 1.7 | 1.7 |
| ethylene diamine disuccinic acid | 4.2 | 4.2 |
| Calcium formate | 0.3 | 0.3 |
| Amphiphilic alkoxylated grease | 4.0 | 4.0 |
| cleaning polymer¹ | | |
| Chlorhydric acid | 0.8 | 0.8 |
| N,N′-(2S,2′S)-1,1′-(dodecane-1,12- | — | 0.80 |
| diylbis(azanediyl))bis(3-methyl-1- | | |
| oxobutane-2,1-diyl)diisonicotinamide | | |
| Water | 35 | 35 |
| Minors (antioxidant, sulfite, aesthetics, . . . ) | 0.7 | 0.7 |
| Buffers (monoethanolamine) | Up to pH 8 | Up to pH 8 |
| Solvents (1,2-propanediol, ethanol, . . . ) | Up to 100 | Up to 100 |

The robustness of the unit dose articles against water “sweating” through the film was measured via the water condensation test:

| | Comparative | |
|---|---|---|
| | Example 2 | Example 2 |
| % unit dose articles that present | 0% | 0% |
| condensation after 3 days at 35° C. | | |
| % unit dose articles that present | 4.8% | 0% |
| condensation after 15 days at 35° C. | | |
| % unit dose articles that present | 14.4% | 0% |
| condensation after 30 days at 35° C. | | |

Thus, it is clear that the di-amido gellant is able to improve binding the water within the fluid composition, and hence prevent leakage of water through the film.

The unit dose articles of examples 3 to 5 were prepared using the method of comparative example 1, and example 1 of the present invention, however using different volumes of fluid composition:

| | Example 3 | Example 4 | Example 5 |
|---|---|---|---|
| Amount of fluid composition in | 40 mL | 35 mL | 31 mL |
| the unit dose article | | | |
| Ingredients | Weight % | | |
| C₁₁₋₁₆ Alkylbenzene sulfonic acid | 18.0 | 12.5 | 19.0 |
| C₁₂₋₁₄ Alkyl sulfate | — | 2.0 | — |
| C₁₂₋₁₄ alkyl 7-ethoxylate | 17.0 | 17.0 | 16.0 |
| C₁₂₋₁₄ alkyl ethoxy 3 sulfate | 7.5 | — | 7.0 |
| Citric acid | 3.5 | 1.0 | 2.0 |
| Chloryhidric acid | — | 0.8 | 0.3 |
| C₁₂₋₁₈ Fatty acid | 10.0 | 17.0 | 15.0 |
| Sodium citrate | — | 4.0 | — |
| enzymes | 0-3.0 | 0-3.0 | 0-3.0 |
| Ethoxylated Polyethylenimine² | 2.2 | — | — |
| Hydroxyethane diphosphonic acid | 0.6 | 0.5 | 2.2 |
| Amphiphilic alkoxylated grease | 2.5 | — | 3.5 |
| cleaning polymer¹ | | | |
| Ethylene diamine tetra(methylene | — | — | 0.4 |
| phosphonic) acid | | | |
| Brightener | 0.2 | 0.3 | 0.3 |
| Perfume microcapsules³ | 0.4 | — | — |
| Water | 20.5 | 22.5 | 15.5 |
| Calcium chloride | — | — | 0.01 |
| Perfume | 1.7 | 0.6 | 1.6 |
| N,N′-(2S,2′S)-1,1′-(dodecane- | 0.30 | 0.28 | — |
| 1,12-diylbis(azanediyl))bis(3- | | | |
| methyl-1-oxobutane-2,1-diyl) | | | |
| diisonicotinamide | | | |
| N-[(1S)-2-methyl-1-[8-[[(2S)-3- | — | — | 0.35 |
| methyl-2-(pyridine-4- | | | |
| carbonylamino)pentanoyl]amino] | | | |
| octylcarbamoyl]butyl]pyridine- | | | |
| 4-carboxamide | | | |
| Minors (antioxidant, sulfite, | 2.0 | 4.0 | 2.3 |
| aesthetics, . . . ) | | | |

| | |
|---|---|
| Buffers (monoethanolamine) | To pH 8.0 |
| Solvents (1,2 propanediol, ethanol) | To 100 parts |

²Polyethylenimine (MW = 600) with 20 ethoxylate groups per —NH.
³Perfume microcapsule slurry comprising 60% by weight water.

A wash residue test on example 3 was performed, using the method described above, rating an average grade of 1 in the test.

The following are examples of multicompartment unit dose articles wherein the liquid composition is enclosed within a PVA film (Monosol M8630, having a thickness of 76 μm).

| | Example 6 | | | Example 7 | | |
|---|---|---|---|---|---|---|
| Compartment | 1 | 2 | 3 | 1 | 2 | 3 |
| Amount of | 34 | 3.5 | 3.5 | 25 | 1.5 | 4 |
| composition in each | | | | | | |
| compartment (mL) | | | | | | |
| Ingredients | Weight % | | | | | |
| C₁₁₋₁₆ Alkylbenzene | 18 | 20 | 20 | 18 | 23 | 28 |
| sulfonic acid | | | | | | |
| C₁₂₋₁₄ alkyl 7-ethoxylate | 17 | 17 | 17 | 17 | 15 | 10 |
| C₁₂₋₁₄ alkyl ethoxy | 5.5 | 7.5 | 6 | 6 | 6 | |
| 3 sulfate | | | | | | |
| Citric acid | 0.5 | | 2 | | | 2 |
| C₁₂₋₁₈ Fatty acid | 13 | 13 | 13 | 16 | 8 | 12 |
| 4-Formyl Phenyl | — | — | — | 0.03 | — | — |
| Boronic Acid | | | | | | |
| Ethoxylated | 2.2 | 2.2 | 2.2 | — | — | — |
| Polyethylenimine² | | | | | | |
| Hydroxyethane | 0.6 | 0.6 | 0.6 | — | 2.2 | |
| diphosphonic acid | | | | | | |
| Ethylene diamine tetra | — | — | — | 0.4 | — | — |
| (methylene phosphonic) | | | | | | |
| acid | | | | | | |
| Amphiphilic | 3.5 | — | — | 2.5 | — | — |
| alkoxylated grease | | | | | | |
| cleaning polymers¹ | | | | | | |
| Brightener | 0.2 | 0.2 | 0.2 | 0.3 | — | — |
| Perfume microcapsules | 0.4 | — | — | | — | — |
| Protease (40.6 mg/g/)⁴ | — | 2 | — | 1.0 | — | — |
| Natalase 200 L | 0.15 | — | — | 0.2 | — | — |
| (29.26 mg/g)⁵ | | | | | | |
| Termamyl Ultra | 0.1 | — | — | 0.1 | — | — |
| (25.1 mg/g)⁵ | | | | | | |
| Mannaway 25 L | 0.1 | — | — | 0.1 | — | — |
| (25 mg/g)⁵ | | | | | | |
| Lipase (16.91 mg/g) | — | — | 0.5 | — | — | — |
| Lipolex ® | — | — | — | 0.4 | — | — |
| Whitezyme | 0.1 | — | — | 0.1 | — | — |
| (20 mg/g)⁵ | | | | | | |
| Fluorescent | 0.2 | — | — | 0.2 | — | — |
| Whitening Agent | | | | | | |
| Phenyl Boronic Acid | — | 0.04 | — | | — | — |
| N,N′-(2S,2′S)-1,1′- | 0.35 | — | — | 0.2 | — | — |
| (dodecane-1,12- | | | | | | |
| diylbis(azanediyl))bis | | | | | | |
| (3-methyl-1- | | | | | | |
| oxobutane-2,1-diyl) | | | | | | |
| diisonicotinamide | | | | | | |
| N,N′-(2S,2′S)-1,1′- | — | 0.2 | 0.2 | 0.2 | — | — |
| (propane-1,3- | | | | | | |
| diylbis(azanediyl))bis | | | | | | |
| (3-methyl-1- | | | | | | |
| oxobutane-2,1-diyl) | | | | | | |
| diisonicotinamide | | | | | | |
| Water | 21.5 | 15.5 | 15.5 | 22 | 8 | 9 |
| CaCl₂ | — | — | — | — | 0.01 | — |
| Perfume | 1.7 | 1.7 | — | 1.5 | 0.5 | — |
| Hydrogenated | 0.1 | | — | — | — | — |
| castor oil | | | | | | |
| Minors (antioxidant, | 2 | 2 | 2 | 2.2 | 2.2 | 2 |
| sulfite, | | | | | | |
| aesthetics, . . . ) | | | | | | |

| | |
|---|---|
| Buffer | To pH 8 |
| (monoethanolamine) | |
| Solvents | To 100 parts |
| (1,2 propanediol, | |
| ethanol) | |

⁴Available from Genencor International, South San Francisco, CA.
⁵Available from Novozymes, Denmark.

The following are examples of unit dose articles wherein the liquid composition is enclosed within a PVA film (Monosol M8630, having a thickness of 76 μm).

| | Example 8 | Example 9 |
|---|---|---|
| Amount of fluid composition | 25 mL | 25 mL |
| unit does article | | |
| Ingredients | Weight % | |
| Monoethanolamine: C₁₂₋₁₅ EO•3•SO₃H | 37.0 | 35.0 |
| Monoethanolamine: C₁₆₋₁₇ highly | 5.9 | 6.0 |
| soluble alkyl sulfate | | |
| C₁₂₋₁₄ dimethylamine-N-oxide | 1.7 | 1.7 |
| Ethoxylated Polyethyleneimine² | 3.9 | 4.0 |
| Citric acid | — | 2.0 |
| Amphiphilic alkoxylated grease | 3.9 | 2.5 |
| cleaning polymer¹ | | |
| C₁₂₋₁₈ Fatty acid | 3.0 | — |
| Suds suppression polymer | 0.1 | 0.1 |
| C₁₁₋₈ HLAS | 13.4 | 10.0 |
| HEDP | — | 1.0 |
| Tiron (1,2-dihydroxybenzene-3,5- | 2.0 | — |
| disulphonic acid) | | |
| Brightener | 0.1 | 0.2 |
| Perfume microcapsules | 5.0 | — |
| Water | 21 | 25 |
| Perfume | 1.5 | 1.7 |
| N,N′-(2S,2′S)-1,1′-(octane-1,8- | 0.4 | 0.35 |
| diylbis(azanediyl))bis(1-oxo-3- | | |
| phenylpropane-2,1-diyl)diisonicotinamide | | |
| Minors (antioxidant, sulfite, aesthetics, . . . ) | 1.5 | 1.5 |

| | |
|---|---|
| Buffers (monoethanolamine) | To pH 8.0 |
| Solvents (1,2 propanediol, ethanol) | To 100 parts |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A unit dose article comprising a water soluble film encapsulating a fluid composition, wherein the fluid composition comprises:
a. a di-amido gellant; and
b. from about 11 wt % to about 70 wt % by weight of water.

2. The unit dose article according to claim 1, wherein the di-amido gellant is present at a level of from about 0.01 wt % to about 10 wt % by weight of the fluid composition.

3. The unit dose article according to claim 1, wherein the di-amido gellant has the following formula: 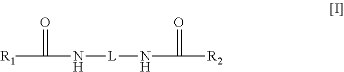
wherein: R₁ and R₂ are aminofunctional end-groups which may be the same or different, and L is a linking moiety of molecular weight from about 14 to about 500 g/mol;

4. The unit dose article according to claim 1, wherein the di-amido gellant comprises a pH tuneable group, such that the di-amido gellant has a pKa of from about 0 to about 30.

5. The unit dose article according to claim 1, wherein the fluid composition further comprises a surfactant.

6. The unit dose article according to claim 1, wherein the di-amido gellant has a molecular weight from about 150 to about 1500 g/mol in the fluid composition.

7. The unit dose article according to claim 1, wherein the di-amido gellant has a minimum gelling concentration (MGC) of from about 0.1 to about 100 mg/mL.

8. The unit dose article according to claim 1, wherein the fluid composition comprises:
a. from about 0.0001% to about 8% by weight of a detersive enzyme; and
b. a neat pH from about 6.5 to about 10.5.

9. The unit dose article according to claim 8, wherein the detersive enzyme is selected from the group consisting of: lipase, protease, cellulase, amylase, mannanase, pectate lyase, xyloglucanase, and mixtures thereof.

10. The unit dose article according to claim 1, wherein the fluid composition comprises:
a. from about 0.1% to about 12% by weight of the bleach or bleach system, and
b. a neat pH of from about 6.5 to about 10.5;
provided that if the fluid composition comprises an enzyme, the bleach active is at least partially physically separated from the enzyme.

11. The unit dose article according to claim 10, wherein if the fluid composition comprises an enzyme, the bleach active is fully separated from the enzyme.

12. The unit dose article according to claim 1, wherein the fluid composition comprises:
a. from about 0.1% to about 12% by weight of a bleach or bleach system, and
b. a neat pH of from about 2 to about 6.

13. The unit dose article according to claim 12, wherein the bleach or bleach system is a peroxide bleach.

14. The unit dose article according to claim 1, wherein the fluid detergent composition further comprises a perfume delivery system selected from the group consisting of: perfume microcapsules, pro-perfumes, polymer particles, functionalized silicones, and mixtures thereof.

15. The unit dose article according to claim 1, wherein the water soluble film material comprises: polyvinyl alcohols, polyvinyl alcohol copolymers and hydroxypropyl methyl cellulose (HPMC), and combinations thereof.

16. A unit dose article comprising a water soluble film encapsulating a fluid composition, wherein the fluid composition comprises:
a. from about 0.075 wt % to about 2 wt % by weight of a di-amido gellant;
b. from about 10% to about 50% by weight of a surfactant; and
c. from about 11 wt % to about 70 wt % by weight of water.
wherein the di-amido gellant has the following formula: 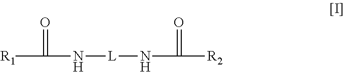
wherein: R₁ and R₂ are aminofunctional end-groups which may be the same or different, and L is a linking moiety of molecular weight from about 14 to about 500 g/mol, and the water soluble film material comprises: polyvinyl alcohols, polyvinyl alcohol copolymers and hydroxypropyl methyl cellulose (HPMC), and combinations thereof.

17. The unit dose article according to claim 16, further comprising a detersive enzyme, selected from the group consisting of: lipase, protease, cellulase, amylase, mannanase, pectate lyase, xyloglucanase, and mixtures thereof.

18. The unit dose article according to claim 16, wherein the fluid detergent composition further comprises a perfume delivery system selected from the group consisting of: perfume microcapsules, pro-perfumes, polymer particles, functionalized silicones, and mixtures thereof.

19. A process of making a unit dose article according to claim 1, comprising the steps of:
(a) making a di-amido gellant premix comprising a di-amido gellant and a solvent;
(b) combining the di-amido gellant premix with a fluid feed, wherein the fluid feed comprises from about 10% to about 70% by weight water, to form a fluid composition; and
(c) encapsulating the fluid composition in a water soluble film.

20. The process according to claim 19, wherein:
(i) in step (a), the di-amido gellant is a pH tuneable di-amido gellant, and the di-amido gellant premix is at a pH such that the pH tuneable di-amido gellant is in an ionic, non-viscosity building, form;
